(19) United States
(12) Patent Application Publication
(10) Pub. No.: US 2026/0015303 A1
(43) Pub. Date: Jan. 15, 2026

(54) METHODS AND SYSTEMS FOR MAKING LIGHT OLEFINS AND ETHANOL FROM A CARBON SOURCE GAS

(71) Applicant: Air Company Holdings, Inc., Brooklyn, NY (US)

(72) Inventors: Lubo Zhou, Deer Park, IL (US); Stafford W. Sheehan, Tiverton, RI (US); Richard Isherwood, Weehawken, NJ (US); Chi Chen, Shrewsbury, MA (US); Qiyuan Wu, Jersey City, NJ (US)

(21) Appl. No.: 19/266,697

(22) Filed: Jul. 11, 2025

Related U.S. Application Data
(60) Provisional application No. 63/670,380, filed on Jul. 12, 2024.
(60) Provisional application No. 63/766,453, filed on Mar. 4, 2025.

(51) Int. Cl. C07C 7/00; C07C 29/17

(52) U.S. Cl. CPC C07C 7/005; C07C 29/172

(57) ABSTRACT

Provided herein are systems and methods for converting carbon dioxide into ethanol and/or light olefins, such as ethylene and propylene. The systems and methods may be utilized to increase the yield of diverse and usable products by converting an otherwise undesirable light hydrocarbon stream, e.g., from a process for converting carbon dioxide to fuel.

### CROSS REFERENCE TO RELATED APPLICATIONS

This application claims the benefit of priority of U.S. Provisional Application No. 63/766,453, filed on Mar. 4, 2025, and U.S. Provisional Application No. 63/670,380, filed on Jul. 12, 2024. The entire contents of each of the foregoing applications is incorporated by reference herein.

### BACKGROUND

As the concentration of carbon dioxide in the atmosphere increases, it is advantageous to develop technologies that remove or mitigate carbon dioxide emissions. As such, development of technologies in various sectors that afford decreased CO₂ emissions has been a priority. A need remains for the development of commercially valuable chemicals and products from carbon dioxide to reduce atmospheric emissions and to eliminate the need for burning fossil fuels to create such chemicals and products.

To achieve the goal of net-zero emissions by 2050, there must be a significant reduction in fossil fuel dependence, as well as significant progress in the use of renewable energy in the field of power generation. There are many fields in which fossil resources cannot easily be replaced by electrification or energy saving technology. For example, materials, such as resins and plastics, fall into this category. Resins and plastics are produced from lower olefins (ethylene, propylene, etc.) derived from naphtha, which is refined from crude oil, or ethane in natural gas. Renewable energy sources such as biomass are beginning to be utilized as alternatives to fossil resources, but these are difficult to supply in sufficient quantities, so the alternatives to fossil resources are presently limited.

The intensity of CO₂ emissions in the production of ethylene, a typical lower olefin, from naphtha is estimated to be 1.8 to 2.0 t-CO₂ per ton of ethylene. Ren, T., et al., “Olefins from conventional and heavy feed stocks: Energy use in steam cracking and alternative processes,” Energy 2006, 31, pp. 425-451. The CO₂ emissions intensity is relatively low at 1.0 to 1.2 t-CO₂ when ethane in natural gas is used as feedstock, but it is estimated to be approximately 10 t-CO₂ when coal is used as feedstock, which results in a very high CO₂ emissions intensity. Resins and plastics are currently produced worldwide at an annual volume of approximately 400 million tons, and this volume is expected to increase. The carbon footprint of resin and plastic production can be significantly reduced by using lower olefins made from recycled CO₂.

### SUMMARY

Disclosed herein is a method of making ethylene and/or ethanol from a carbon source gas comprising: contacting a reduction gas and the carbon source gas with a reduction catalyst to afford an effluent stream comprising a medium hydrocarbon product mixture and a light product mixture, wherein the light product mixture comprises one or more C₁₋₂ hydrocarbons, and CO₂; separating the light product mixture from the medium hydrocarbon product mixture; removing CO₂ from at least a portion of the light product mixture to provide a light hydrocarbon stream comprising the one or more C₁₋₂ hydrocarbons; separating the light hydrocarbon stream into a C₂ stream and a deethanized gas stream; separating the C₂ stream into ethylene and ethane; and optionally contacting the ethylene with an ethylene hydration catalyst to make ethanol. The method may include dividing the light product mixture into a first recycle stream and a second stream; and removing CO₂ from the second stream to provide the light hydrocarbon stream. The method may include chilling and/or drying the light hydrocarbon stream. The method may include separating a second recycle stream from the light hydrocarbon stream, wherein the second recycle stream comprises methane.

Another method is directed to making light olefins (e.g., ethylene and propylene) from a carbon source gas. That method may comprise: contacting a reduction gas and the carbon source gas with a reduction catalyst to afford an effluent stream comprising a medium hydrocarbon product mixture and a light product hydrocarbon mixture; separating the light product mixture from the medium hydrocarbon product mixture; removing CO₂ from at least a portion of the light product mixture to provide a light hydrocarbon stream comprising the one or more C₁₋₂ hydrocarbons; separating the light hydrocarbon stream into a C₂ stream and a deethanized stream; separating the deethanized stream into a C₃ stream and a heavy gas stream; separating the C₃ stream into propylene and propane; removing heavy oil from the medium hydrocarbon product mixture to provide a medium hydrocarbon stream; contacting the medium hydrocarbon stream and the separated heavy gas stream with a cracking catalyst to afford a mixed hydrocarbon stream. The method may include combining the mixed hydrocarbon stream with the effluent stream. The C₂ stream may include ethylene and ethane, and the methos may include optionally processing at least a portion of the ethylene to make ethanol, for example through ethylene hydration.

A system for the production of ethylene and/or ethanol from a carbon source gas is disclosed. That system may comprise: a reduction gas feed; a carbon source gas feed; a reduction reactor comprising a reduction catalyst; a first separator having an effluent stream inlet coupled to the effluent stream outlet, said first separator being configured to separate a light product mixture from a medium hydrocarbon product mixture; a CO₂ removal unit coupled to the first separator; a demethanizer column coupled to the CO₂ removal unit; a deethanizer column coupled to the demethanizer column; a C₂ splitter coupled to the deethanizer column; and an ethylene hydration reactor comprising an ethylene hydration catalyst and configured to receive ethylene from the C₂ splitter. The reduction reactor may have a reduction gas feed inlet, a carbon source feed inlet, and an effluent stream outlet. The system may further comprise a splitter configured to divide the light product mixture into a first recycle stream and a second stream. The CO₂ removal unit may be configured to receive and remove CO₂ from the second stream. The CO₂ removal unit may comprise an amine wash and a stripper column. The system may further comprise a dryer having an inlet coupled to the CO₂ removal unit, and a light hydrocarbon stream outlet, and/or a chiller coupled to the dryer and to the demethanizer column, and/or an ethanol purification unit.

A system for the production of light olefins from a carbon source gas is also disclosed. That system may comprise: a reduction gas feed; a carbon source gas feed; a reduction reactor comprising a reduction catalyst; a first separator having an effluent stream inlet coupled to the effluent stream outlet, said first separator being configured to separate a light product mixture from a medium hydrocarbon product mixture; a CO₂ removal unit coupled to the first separator; a demethanizer column coupled to the CO₂ removal unit; a deethanizer column coupled to the demethanizer column; a C₂ splitter coupled to the deethanizer column; a depropanizer column coupled to the deethanizer column; and a C₃ splitter coupled to the depropanizer column. The system may further comprise: a C₉/C₁₀ splitter coupled to the first separator; and a cracking reactor coupled to the C₉/C₁₀ splitter. The cracking reactor may be configured to receive a medium hydrocarbon stream comprising C₄₋₉ hydrocarbons, and a heavy gas stream from the depropanizer column.

### BRIEF DESCRIPTION OF THE FIGURES

FIG. 1 is a process flow diagram showing conversion of CO₂ and hydrogen to ethanol via ethylene hydration.

FIG. 2 is a process flow diagram showing conversion of CO₂ and hydrogen to ethylene and propylene.

### DETAILED DESCRIPTION

Disclosed herein are methods of making ethanol and methods of making light olefins from at least a portion of a light product mixture generated from CO and/or CO₂ hydrogenation. The methods and systems disclosed herein may be incorporated into, or otherwise used in combination with, any method or system including supplying a carbon source gas and a reduction gas to a reactor for CO and/or CO₂ hydrogenation that produces an effluent comprising paraffins, olefins, or a combination thereof, including, but not limited to those methods and systems disclosed in co-owned U.S. Patent Application Publication No. 2024/0124792, published on Apr. 18, 2024, titled: SYNTHETIC FUELS, AND METHODS AND APPARATUS FOR PRODUCTION THEREOF; or in co-owned International Publication No. WO 2025/096891, published on May 8, 2025, titled: SYSTEMS, METHODS, AND CATALYSTS FOR THE PRODUCTION OF SUSTAINABLE AVIATION FUEL; the entire contents of the foregoing applications are incorporated by reference herein.

Ethanol made as a co-product by the processes and systems disclosed have a lower CAPEX than previously contemplated methods of thermochemically converting carbon dioxide into ethanol.

The methods disclosed herein offer several economical and technical advantages:
Reduces catalyst deactivation rate due to the CO₂ co-feeding;
Improves conversion/selectivity by consuming any hydrogen formed from the dehydrogenation reaction;
Eliminates the use of complicated separation techniques for removing oxygenates, i.e., under the conditions disclosed herein, oxygenates will be converted to hydrocarbons;
Converts CO₂ to useful products;
Consolidates multiple reactions (i.e., dehydrogenation, cracking, hydrogenation, and oxygenates condensations to hydrocarbons) into one cracking reactor.
   Methods of Making Ethanol and/or Ethylene

A method of making ethylene, ethanol or both ethylene and ethanol from a carbon source gas comprising: contacting a reduction gas and the carbon source gas with a reduction catalyst to afford an effluent stream comprising a medium hydrocarbon product mixture and a light product mixture, wherein the light product mixture comprises one or more C₁₋₂ hydrocarbons, and CO₂; separating the light product mixture from the medium hydrocarbon product mixture; removing CO₂ from the light product mixture to provide a light hydrocarbon stream comprising the one or more C₁₋₂ hydrocarbons; separating the light hydrocarbon stream into a C₂ hydrocarbon stream and a deethanized stream; separating the C₂ hydrocarbon stream into ethylene and ethane; and optionally contacting the ethylene with an ethylene hydration catalyst to make ethanol.

A method of making ethanol from a carbon source gas is also disclosed. That method comprises: contacting a first reduction gas and the carbon source gas with a reduction catalyst to afford an effluent stream comprising a medium hydrocarbon product mixture and a light product mixture; separating the light product mixture from the medium hydrocarbon product mixture; removing CO₂ from the light product mixture to provide a light hydrocarbon stream comprising the one or more C₁₋₂ hydrocarbons; separating the light hydrocarbon stream into a C₂ hydrocarbon stream and a deethanized stream; separating the C₂ hydrocarbon stream into ethylene and ethane; and contacting the ethylene with an ethylene hydration catalyst to make ethanol. The deethanized stream may comprise C₃₊ olefins and/or paraffins.

The light product mixture may comprise one or more C₁₋₂ hydrocarbons, and CO₂. The light product mixture may comprise one or more C₁₋₂ hydrocarbons, CO₂, CO, and/or H₂. The first recycle stream and the second stream contain the same components in the same percentages as the light product mixture. That is, the first recycle stream and the second stream may comprise one or more C₁₋₂ hydrocarbons, CO₂, CO, and/or H₂. The second stream may comprise about 1 wt % to about 99.9 wt % of the light product mixture, with the remaining amount being the first recycle stream.

The medium hydrocarbon product mixture may comprise one or more C₃₋₉ paraffins and/or olefins. The medium hydrocarbon product mixture may comprise one or more C₄₋₉ paraffins and/or olefins. The medium hydrocarbon product mixture may comprise one or more C₃₋₉ paraffins and/or olefins, and one or more C₁₀₋₁₆ paraffins and/or olefins. The medium hydrocarbon product mixture may comprise one or more C₄₋₉ paraffins and/or olefins, and one or more C₁₀₋₁₆ paraffins and/or olefins. One of ordinary skill in the art will readily understand that the composition of the medium hydrocarbon product mixture may be varied by the selection of reduction catalyst and operating conditions of the reduction reactor. One of ordinary skill in the art will readily understand that the medium hydrocarbon product mixture may be further processed by any means known in the art for downstream use.

The step of contacting the first reduction gas and the carbon source gas with the reduction catalyst may occur at a paraffin temperature which may be at least 80° C., or at least 100° C., or at least 120° C. The paraffin temperature may be 550° C. or less, or 600° C. or less, or 650° C. or less. The paraffin temperature may be from about 100° C. to about 600° C. The paraffin temperature may be from about 200° C. to about 500° C., about 300° C. to about 500° C., about 350° C. to about 500° C., or about 350° C. to about 400° C. The paraffin temperature may be about 325° C., about 350° C., about 375° C., about 400° C., or about 425° C.

In certain embodiments, contacting the first reduction gas and the carbon source gas with the reduction catalyst occurs at a paraffin pressure from about 50 psi to about 4000 psi. The paraffin pressure may be about 75 psi to about 500 psi, about 100 psi to about 475 psi, about 100 psi to about 450 psi, about 125 psi to about 425 psi, about 125 psi to about 400 psi, about 200 psi to about 400 psi, or about 75 psi to about 225 psi. The paraffin pressure may be about 75 psi, about 100 psi, about 125 psi, about 150 psi, about 175 psi, about 200 psi, about 225 psi, about 250 psi, about 350 psi, about 400 psi, about 450 psi, or about 500 psi.

In certain embodiments, each of the light product mixture, and the medium hydrocarbon product mixture comprises a mixture of olefins and paraffins. The ratio of olefins to paraffins in each of the light product mixture and/or the medium hydrocarbon product mixture may be at least about 1:1, with the amount of olefins being about equal to or more than the amount of paraffins present therein. The ratio of olefins to paraffins in each of the light product mixture and/or the medium hydrocarbon product mixture may be at least about 2:1, at least about 3:1, at least about 4:1, at least about 5:1, at least about 6:1, at least about 7:1, at least about 8:1, at least about 9:1, or at least about 10:1. The ratio of olefins to paraffins in each of the light product mixture and/or the medium hydrocarbon product mixture may be about 1:1 to about 20:1, about 5:1 to about 20:1, or about 5:1 to about 15:1.

The method may further comprise: removing CO₂ from the light product mixture to provide a light hydrocarbon stream comprising one or more C₁₋₂ hydrocarbons; separating C₂ hydrocarbons from the light hydrocarbon stream; and separating ethylene from ethane. The method may also include contacting the ethylene with water and an ethylene hydration catalyst to make ethanol.

The light product mixture may be divided into a second stream and a first recycle stream. In certain embodiments, the method includes removing CO₂ from the second stream to provide the light hydrocarbon stream. The light hydrocarbon stream may further comprise C₃ hydrocarbons.

There may be unconverted CO₂ in the light product mixture or the second stream which may preferably be removed to avoid downstream dry ice generation. The step of removing CO₂ may comprise contacting the light product mixture or the second stream with an amine wash to remove CO₂. The method may include regenerating the amine in a stripper column coupled to the amine wash. Other means of separating CO₂ from a light product mixture may also be used. In a particular embodiment, the CO₂ will be absorbed by the amine, and will be released from the amine in the stripper column. The method may also include combining the removed CO₂ with the first recycle stream and/or the second recycle stream and/or with the carbon source gas.

The method includes separating C₂ hydrocarbons from the light hydrocarbon stream by providing the light hydrocarbon stream through a de-ethanizer, or other means known in the art. C₂ hydrocarbons (i.e., ethylene and ethane) will rise to the top of the de-ethanizer column, thereby being separated from the remainder of the stream. The remainder of the stream, or “deethanized stream,” which may comprise C₃₊ olefins and/or paraffins, may be combined with the medium hydrocarbon product mixture, other hydrocarbon streams or a downstream upgrade unit for downstream processing (e.g., for conversion to sustainable aviation fuel). The downstream upgrade unit may be, but is not limited to, one or more of the following processes/systems: aromatization, oligomerization, or hydrogenation to make sustainable aviation fuel.

After C₂ hydrocarbons are isolated, ethylene may be separated from ethane by any means known in the art. For example, ethylene may be separated from ethane by using a C₂ splitter distillation column (also referred to herein as a “C₂ splitter”). A C₂ splitter may also be known in the art as an “ethylene splitter” and is commonly operated at high-pressure, utilizing closed-cycle propylene, ethylene or mixed refrigeration, though it may also operate at low or medium pressure. The design of C₂ splitter may be influenced by factors, including process requirements, economics and safety.

The method may include contacting ethylene and water with an ethylene hydration catalyst at an ethylene hydration temperature and an ethylene hydration pressure to produce ethanol.

The ethylene separated from the C₂ hydrocarbon stream may be fed into an ethylene hydration reactor. The ethylene hydration reactor may be a fixed bed flow reactor, or other type of reactor. In the ethylene hydration reactor, the ethylene may be mixed with steam at a ratio of about 0.6 H₂O:C₂—H₄, though the ratio may vary depending on the catalyst used. The steam and ethylene may be heated to a temperature of about 100′C. to about 300° C., about 150° C., to about 300° C., about 200° C. to about 300° C., about 210° C., to about 270° C., or about 220° C. to about 260° C. The steam and ethylene may be heated to a temperature of about 250° C., for phosphoric acid on silica catalysts.

In certain embodiments, the method comprises: (i) separating ethylene from the C₂ hydrocarbon stream; and (ii) hydrating the ethylene. The step of hydrating may comprise contacting the ethylene with water and an ethylene hydration catalyst at an ethylene hydration temperature and an ethylene hydration pressure to produce ethanol.

Catalysts for ethylene hydration which are suitable for the presently disclosed systems and methods are disclosed in the following patents, each of which is incorporated by reference in its entirety: U.S. Pat. Nos. 1,873,536; 3,452,106; and 4,482,767.

Catalysts for the ethylene hydration reaction, which are referred to in as the “ethylene hydration catalyst,” may be any suitable acid catalyst (solid or liquid), molecular weight sieve (e.g., MWW family), or zeolite. The ethylene hydration catalyst may be selected from the group consisting of: phosphoric acid on silica; phosphoric acid on carbon; zeolites, such as H-ZSM-5 or H-Beta; acidicpolysiloxanes; polyturngstate acids; metal phosphides, such as germanium, titanium, tin, and silicon phosphide; and any combination thereof. The ethylene hydration catalyst may be a resin-type catalyst or solid phosphoric acid (SPA) catalyst. In further embodiments, the ethylene hydration catalyst is a sulfonated styrene-divinylbenzene copolymer resin or phosphoric acid on silica gel. The ethylene hydration catalyst may be selected from a group consisting of: a crystalline zeolite, a silicoaluminophosphate, and any combination thereof.

The ethylene hydration catalyst may a zeolite. The ethylene hydration catalyst may be selected from a group consisting of: SAPO-5, H-SAPO-34, ZSM-11, TNU-9, IM-5, ZSM-35, ZSM-22, ZSM-23, SSZ-13, UZM-12, UZM-9, UZM-5, RUB-13, ZSM-5, ZSM-34, and any combination thereof.

The ethylene hydration temperature may be from about 100° C. to about 400° C., about 100° C. to about 250° C., about 200° C. to about 400° C., about 150° C. to about 300° C., about 300° C. to about 400° C., or about 250° C. to about 350° C. The ethylene hydration temperature may be about 100° C., about 200° C., about 250° C., about 300° C., or about 400° C.

The ethylene hydration pressure may be from about 500 psi to about 1500 psi, about 600 psi to about 1400 psi, about 700 psi to about 1300 psi, about 500 psi to about 1000 psi, about 900 psi to about 1500 psi, about 800 psi to about 1000 psi, or about 1100 psi to about 1500 psi.

The method may further comprise drying the light hydrocarbon stream to remove water. A molecular sieve bed or any other means known for use in the art may be used to dry the light hydrocarbon stream. Drying may be performed after removing CO₂ and before cooling, when present, and before removing methane from the stream.

The method may comprise cooling the light hydrocarbon stream. The method may comprise drying the light hydrocarbon stream to remove water, followed by cooling the light hydrocarbon stream. The step of cooling may be followed by separating a second recycle stream from the cooled light hydrocarbon stream. Separating the second recycle stream may be accomplished by providing the cooled light hydrocarbon stream through a demethanizer column. In the demethanizer column, methane and all lighter components, such as CO and H₂, will be removed from the cooled light hydrocarbon stream and exit through the top of the column. The removed gas stream, referred to as a second recycle stream, may be combined with the first recycle stream and recycled into the feed and optionally, in part, combined with a purge stream to remove inert components, such as methane. The second recycle stream may comprise methane, CO and H₂. The second recycle stream may be combined with the first recycle stream.

In certain embodiments, the method comprises: separating a second recycle stream comprising methane from the cooled light hydrocarbon stream; followed by separating C₂ hydrocarbons to produce a C₂ hydrocarbon stream; followed by, separating ethylene from the C₂ hydrocarbon stream; and optionally hydrating the ethylene.

The method may include combining the first recycle stream, optionally combined with the second recycle stream, with the reduction gas and/or the carbon source gas prior to contacting with the reduction catalyst.

The ethanol produced by the systems and methods herein may be food grade or high purity ethanol.

The method may include a step of purifying the ethanol by any means known in the art to remove impurities from the alcohol. For example, purifying may be accomplished by distillation. Distillation can be accomplished in batch or continuous systems, using fractional distillation, steam distillation, vacuum distillation, short path distillation, zone distillation, or other techniques. Pressure-swing and azeotropic distillation techniques can also be used to purify the ethanol from other impurities. The condensate will ideally be high purity ethanol; however, minor impurities may sometimes be present. The method of purification may comprise contacting the (impure) ethanol with a distillation apparatus, such as one disclosed in U.S. Patent Application Publication No. 2023/0286888, thereby generating pure ethanol. The entire contents of the foregoing patent document is incorporated by reference herein.

The methods and systems herein may be for the production of ethanol as a by-product of another process whereby hydrocarbons are the predominant desired product. In these situations, where ethanol production is a by-product, ethanol production can be significant. The concentration of ethanol in aqueous solution from CO₂ hydrogenation may be at least about 2 wt %, at least about 5 wt %, at least about 10 wt %, or at least about 12 wt %.

Carbon yield of ethanol from the light product mixture (i.e., C₃ effluent from the hydrogenation reaction) may be up to about 50 wt %. The carbon selectivity to ethylene may be about 1 wt % to about 40 wt %, about 10 wt % to about 40 wt %, about 1 wt % to about 30 wt %, about 10 wt % to about 30 wt %, about 1 wt % to about 25 wt %, about 5 wt % to about 25 wt %, or about 10 wt % to about 25 wt %.

### Methods of Making Light Olefins

Disclosed herein are methods of making light olefins from CO and/or CO₂ hydrogenation. Also disclosed are methods of making light olefins, i.e. ethylene and propylene, directly from a carbon source gas, e.g., CO₂, and hydrogen, preferably, though not limited to, green hydrogen, blue hydrogen, or a combination thereof. Light olefins, as used herein, refers to ethylene and propylene.

A method of making light olefins from a carbon source gas comprises: contacting a first reduction gas and the carbon source gas with a reduction catalyst to afford an effluent stream comprising a medium hydrocarbon product mixture and a light product mixture; separating the light product mixture from the medium hydrocarbon product mixture; removing CO₂ from the light product mixture to provide a light hydrocarbon stream comprising one or more C₁₋₃ hydrocarbons; separating the light hydrocarbon stream into a C₂ hydrocarbon stream and a deethanized stream; separating the C₂ hydrocarbon stream into ethylene and ethane; separating the deethanized stream into a C₃ hydrocarbon stream and a heavy gas stream; and separating the C₃ hydrocarbon stream into propylene and propane. The heavy gas stream may comprise C₄₊ hydrocarbons. The heavy gas stream may comprise C₄ hydrocarbons. The heavy gas stream may comprise over about 60 wt %, over about 70 wt %, over about 80 wt %, over about 85 wt %, over about 90 wt %, or over about 95 wt % C₄ hydrocarbons. The heavy gas stream may comprise over about 70 wt % C₄ hydrocarbons, and less than about 30 wt % of C₅₋₈ hydrocarbons. The heavy gas stream may comprise over about 80 wt % C₄ hydrocarbons, and less than about 20 wt % of C₅₋₈ hydrocarbons. The heavy gas stream may comprise over about 90 wt % C₄ hydrocarbons, and less than about 10 wt % of C₅₋₈ hydrocarbons.

The method of making light olefins may further comprise: removing heavy oil from the medium hydrocarbon product mixture to provide a medium hydrocarbon stream; contacting the medium hydrocarbon stream and the heavy gas stream with a cracking catalyst in a cracking reactor to afford a mixed hydrocarbon stream. The heavy oil may comprise C₁₀₊ hydrocarbons.

The method may include combining the mixed hydrocarbon stream with the effluent stream. By combining the mixed hydrocarbon stream back into the effluent stream, the selectivity (or otherwise understood as the yield) to light olefins may be about 10 carbon mole % to about 45 carbon mole %, about 15 carbon mole % to about 40 carbon mole %, about 10 carbon mole % to about 25 carbon mole %, about 20 carbon mole % to about 45 carbon mole %, or about 20 carbon mole % to about 40 carbon mole %.

The step of removing heavy oil from the medium hydrocarbon product mixture may include directing the medium hydrocarbon product mixture through a C₉/C₁₀ splitter configured to separate the medium hydrocarbon product mixture into a medium hydrocarbon stream comprising C₄₋₉ hydrocarbons, and a heavy oil. The heavy oil may comprise C₁₀₊ hydrocarbons. The heavy oil may be processed by any means known in the art. The medium hydrocarbon stream may comprise separated C₄₋₉ hydrocarbons.

In some embodiments, contacting the medium hydrocarbon stream and the heavy gas stream with the cracking catalyst occurs at a cracking temperature from about 400° C. to about 700° C., about 450° C. to about 700° C., or about 500° C. to about 650° C. Contacting the medium hydrocarbon product mixture and the heavy gas stream with the cracking catalyst occurs at a cracking pressure from about 0 psig to about 100 psig.

The mixed hydrocarbon stream may comprise CO, hydrogen and light olefins. The mixed hydrocarbon stream may comprise CO, CO₂, hydrogen, light olefins, and optionally trace amounts of water.

There may be unconverted CO₂ in the light product mixture which may be removed to avoid downstream dry ice generation. The step of removing CO₂ may comprise contacting the light product mixture with an amine wash to remove CO₂. The method may include regenerating the amine in a stripper column coupled to the amine wash. Other means of separating CO₂ from a light product mixture may also be used. The method may also include combining the removed CO₂ with a second recycle stream and/or with the carbon source gas.

The method includes separating C₂ hydrocarbons from the light hydrocarbon stream by providing the light hydrocarbon stream through a deethanizer column, or other means known in the art. C₂ hydrocarbons (i.e., ethylene and ethane) will rise to the top of the deethanizer column and may be separated from the remainder of the stream. The remainder of the stream, or “deethanized stream,” which may comprise C₃₊ olefins and/or paraffins, may be directed to a depropanizer column configured to separate the C₃ hydrocarbons (i.e., propylene and propane) from heavier (C₄₊ hydrocarbons) hydrocarbons, herein referred to as a heavy gas stream. The C₃ hydrocarbon stream may be separated, e.g., in a C₃ splitter, into propylene and propane which may be provided to any storage tank or downstream processing, as desired and readily understood by one of skill in the art.

The method may further comprise drying the light hydrocarbon stream to remove water. A molecular sieve bed or any other means known for use in the art may be used to dry the light hydrocarbon stream. Drying may be performed after removing CO₂ and before cooling, when present, and before removing methane from the stream.

The method may comprise cooling the light hydrocarbon stream, after removing the CO₂. The method may comprise drying the light hydrocarbon stream to remove water, followed by cooling the light hydrocarbon stream. The step of cooling may be followed by separating a second recycle stream from the cooled light hydrocarbon stream. Separating the second recycle stream may be accomplished by providing the cooled light hydrocarbon stream through a demethanizer column. In the demethanizer column, methane and all lighter components, such as CO and H₂ will be removed from the light hydrocarbon stream and exit through the top of the column. The removed gas stream, referred to as a second recycle stream, may be recycled into the feed and optionally, in part, combined with a purge stream to remove inert components, such as methane. The second recycle stream may comprise methane, CO and H₂. The second recycle stream may be combined with the first recycle stream.

In certain embodiments, the method comprises: separating a second recycle stream comprising methane from the cooled light hydrocarbon stream; followed by separating C₂ hydrocarbons to produce a C₂ hydrocarbon stream; followed by separating ethylene from the C₂ hydrocarbon stream.

The method may include combining the second recycle stream with the reduction gas and/or the carbon source gas prior to contacting with the reduction catalyst.

The ethylene, propylene, or both of the light olefins produced by the methods disclosed herein may be subsequently subjected to any oligomerization process or polymerization process known in the art for use with light olefins. In an embodiment, the propylene may be subjected to propylene polymerization to yield polypropylene. In an embodiment, polymerization may be performed by subjecting the ethylene, propylene, or both of the light olefins to a Ziegler-Natta catalyst.

The molar selectivity of CO₂ to light olefins may be greater than about 30%, greater than about 35%, greater than about 40%, greater than about 45%, greater than about 50%, greater than about 55%, greater than 60%, or greater than about 65%. The term “selectivity” refers to “molar selectivity” when used herein, unless specifically defined otherwise. The selectivity of CO₂ to light olefins may be about 30% to about 70%, about 35% to about 70%, about 40% to about 70%, about 45% to about 70%, about 50% to about 70%, about 50% to about 65%, about 50% to about 60%, about 55% to about 70%, about 60% to about 70%, or about 65%. Selectivity is presented in carbon mole %. The molar selectivity for light olefins is determined by adding (molar selectivity for ethylene)+(selectivity for propylene), with each selectivity value calculated according to Equation 1. Referring to Equation 1: Cₓ represents a hydrocarbon having a carbon number of x; Cmol.Cₓ represents mole fraction of Cₓ in the product stream; Cmol.CO_{2feed} represents mole fraction of CO₂ in the feed stream; and Cmol.CO_{2product} represents the mole fraction of CO₂ in the product stream.

$$Selectivity for hydrocarbon {C}_{x} ( {SC}_{x} ) = [ Cmol \cdot {C}_{x} / ( Cmol \cdot {CO}_{2 feed} - Cmol \cdot {CO}_{2 product} ) ] \qquad Eq . 1$$

The selectivity of CO₂ to ethylene may be greater than about 15%, greater than about 20%, greater than 25%, or greater than about 30%. The selectivity of CO₂ to ethylene may be about 15% to about 35%, about 20% to about 35%, about 25% to about 35%, about 27% to about 35%, about 30% to about 35%, or about 30%.

### Systems for Ethanol Production

Provided herein are systems for converting a carbon source gas (e.g. CO and/or CO₂) to ethanol. Certain components of these systems are described as being “coupled” to one another. As will be appreciated, the term “coupled” as used herein describes components that are operationally linked to one another, but does not preclude the presence of intervening components between those said to be coupled to one another. Additionally, as will be appreciated, various system components are described as “having” certain features. For example, in certain embodiments the reduction reactor is described as having a first reduction gas feed inlet, a first carbon source inlet, and a paraffin product outlet. Such descriptions do not preclude, and specifically contemplate, the presence of additional features, such as inlets, outlets, valves, control mechanisms, measurement devices, heating and/or cooling systems, etc. Additionally, in the systems of the present disclosure, certain components are described as having one or more outlets or inlets. Such outlets and inlets may represent separate structural elements, or may be combined into a single inlet or outlet as suitable. The person of ordinary skill in the art will recognize that, once the critical features and operating conditions of systems such as those described herein are understood, the detailed design and operation of such systems involved many choices, such as specific reagent flows, separation steps, etc. While the present disclosure provides a number of specific embodiments, any suitable combination of these design choices may be made.

As used herein, the term “Cₓ hydrocarbon” indicates hydrocarbon molecules having the number of carbon atoms represented by the subscript “x”. The term “Cₓ+ hydrocarbons” indicates those molecules noted above having the number of carbon atoms represented by the subscript “x” or greater. For example, “C₁₀₊ hydrocarbons.” would include C₁₀, C₁₁ and higher carbon number hydrocarbons. Similarly “Cₓ— hydrocarbons” indicates those molecules noted above having the number of carbon atoms represented by the subscript “x” or fewer. The various systems and methods of the present disclosure sometimes reference fractions with particular carbon numbers (e.g., C_{X-Y}). As used herein, “C₅-C₈” means molecules having 5 to 8 carbons. As will be understood, these carbon numbers refer to the carbon makeup of the majority of the fraction, but said fractions may include additional components with carbon numbers that are higher or lower than indicated. Separators which are capable of creating these fractions are well known in the art, and can be adjusted as needed to afford suitable product mixtures as disclosed herein, or as otherwise desired by the operator. Certain components of said system are referred to by numbers in brackets (i.e., [10]).

A system for the production of ethanol is disclosed herein. The systems may include: a reduction gas feed; a carbon source gas feed; a reduction reactor comprising a reduction catalyst; a first separator configured to receive the effluent stream from the reduction reactor, and to separate a light product mixture from a medium hydrocarbon product mixture; a CO₂ removal unit configured to receive and remove CO₂ from at least a portion of the light product mixture; a demethanizer column; a deethanizer column; a C₂ splitter; and an ethylene hydration reactor comprising an ethylene hydration catalyst. The ethylene hydration reactor is configured to convert ethylene to ethanol.

The terms used in this second embodiment have the same meanings as indicated above with respect to the first embodiment.

The reduction reactor may have a reduction gas feed inlet, a carbon source feed inlet, and an effluent stream outlet. The reduction gas feed inlet may be coupled to the reduction gas feed. The carbon source gas feed inlet may be coupled to the carbon source gas feed.

The system may include a splitter configured to divide the light product mixture into a first recycle stream and a second stream; and the CO₂ removal unit configured to receive and remove CO₂ from the second stream. In certain embodiments, the CO₂ removal unit is coupled to the splitter and configured to remove CO₂ from the second stream. The CO₂ removal unit may include a second stream inlet and an outlet. The system may include a dryer configured to remove water from the second stream and provide a light hydrocarbon stream. The dryer may be coupled to and downstream from the CO₂ removal unit. The dryer may have a light hydrocarbon stream outlet, and an inlet coupled to the outlet on the CO₂ removal unit.

The CO₂ removal unit may comprise an amine wash and a stripper column to regenerate the amine. The amine wash may be designed to remove CO₂ to avoid dry ice generation in the downstream cryogenic separation. The amine may be regenerated in the stripper column, and the removed CO₂ may be combined with a first recycle stream and recycled by combining with the carbon source gas feed. The stripper column may have a CO₂ outlet and an amine inlet coupled to an amine outlet on the amine wash.

The system may comprise a chiller configured to cool the light hydrocarbon stream. The chiller may be coupled to a dryer and to a demethanizer column. The chiller may be configured to receive a dried light hydrocarbon stream from the dryer and be configured to supply a cooled light hydrocarbon stream to a demethanizer column. Any chiller known for use in the art may be used herein. In certain embodiments, the chiller may reduce the temperature of light hydrocarbon stream to below about −80° C., or below about −85° C. For such an operation, an ethane chiller or ethane-propane chiller may be used. The pressure of the light hydrocarbon stream entering the demethanizer column may be greater than about 20 barg, or about 20 barg to about 30 barg. The pressure of the light hydrocarbon stream may be about 25 barg, or about 30 barg. The demethanizer column may operate at about 20 barg to about 30 barg, or about 22 barg to about 27 barg. The demethanizer column may operate at about 25 barg, or about 30 barg.

The demethanizer column may be coupled to the chiller, or to the dryer, when present. The demethanizer column may be coupled to a deethanizer column. The demethanizer column may include a cooled light hydrocarbon stream inlet, a methane outlet, and a demethanized stream outlet. The demethanized stream outlet may be coupled to a demethanized stream inlet on the deethanizer column. The demethanizer column may be any such distillation column or other apparatus known for use in the art.

The deethanizer column, or otherwise known as a deethanizer, may be any such distillation column or other apparatus known for use in the art. The deethanizer column may be coupled to the demethanizer column and to the C₂ splitter. In certain embodiments, the deethanizer column may be configured to receive the light hydrocarbon stream from the demethanizer column. The deethanizer column may comprise a light hydrocarbon stream inlet, a deethanized stream outlet, and a C₂ hydrocarbon outlet. In other embodiments, the deethanizer column may be configured to receive the demethanized stream from the demethanizer column. The deethanizer column may comprise a demethanized stream inlet, a deethanized stream outlet, and a C₂ hydrocarbon outlet.

The C₂ splitter may have a C₂ hydrocarbon inlet, an ethylene outlet, and an ethane outlet. The C₂ splitter may be configured to separate ethylene and ethane. The C₂ splitter may be coupled to the deethanizer column and the ethylene hydration reactor. The C₂ splitter may be any column or apparatus known for such use in the art.

The ethylene hydration reactor may be loaded with an ethylene hydration catalyst and may have an ethylene gas feed inlet; a water inlet; and an ethanol outlet. The ethylene hydration reactor may also comprise an unreacted ethylene outlet and a water outlet. The ethylene gas feed inlet may be coupled to an ethylene gas outlet on the C₂ splitter. The water inlet may be coupled to a water treatment unit that processes water generated from the hydrogenation reactor.

The ethylene hydration reactor may be configured such that a mixture of ethylene and steam passes through a fixed-bed reactor loaded with one or more ethylene hydration catalysts.

In further embodiments, the system comprises an ethanol purification unit, which may be a distillation column or unit configured to purify the ethanol. The ethanol purification unit may be coupled to the ethylene hydration reactor, and may be configured to receive ethanol from the ethylene hydration reactor. The ethanoyl purification unit may be any such apparatus known for use in the art,

### Systems for the Production of Light Olefins

A system for the production of light olefins from a carbon source gas is disclosed. That system comprises: a reduction gas feed; a carbon source gas feed; a reduction reactor comprising a reduction catalyst; a first separator having an effluent stream inlet coupled to the effluent stream outlet, said first separator being configured to separate a light product mixture from a medium hydrocarbon product mixture; a CO₂ removal unit coupled to the first separator; a demethanizer column coupled to the CO₂ removal unit; a deethanizer column coupled to the demethanizer column; a C₂ splitter coupled to the deethanizer column; a depropanizer column coupled to the deethanizer column; and a C₃ splitter coupled to the depropanizer column.

The system optionally may further comprise: a C₉/C₁₀ splitter coupled to the first separator; and a cracking reactor coupled to the C₉/C₁₀ splitter. The cracking reactor may be configured to receive a medium hydrocarbon stream comprising C₄₋₉ hydrocarbons from the C₉/C₁₀ splitter and the heavy gas stream from the depropanizer column. The cracking reactor may include a heavy gas stream inlet, a medium hydrocarbon stream inlet and a mixed hydrocarbon stream outlet.

The depropanizer column is configured to separate C₃ hydrocarbons from the remainder of the deethanized stream. The depropanizer column may include a deethanized stream inlet, a heavy gas stream outlet and a C₃ hydrocarbon outlet. The deethanized stream inlet may be coupled to the deethanized stream outlet on the deethanizer column. The heavy gas stream outlet may be coupled to the heavy gas stream inlet on the cracking reactor. Any depropanizer column suitable for this purpose may be used with the system disclosed herein. The cracking reactor may be a fluidized bed or fixed bed reactor.

The demethanizer column and the deethanizer column may be connected in series. The demethanizer column, the deethanizer column, and the depropanizer column may be connected in series.

A C₃ splitter may also be known in the art as an “propylene splitter” and is commonly operated at high-pressure, utilizing closed-cycle propylene, ethylene or mixed refrigeration, though it may also operate at low or medium pressure. The design of C₃ splitter may be influenced by factors, including process requirements, economics and safety, and will be readily envisioned by one of ordinary skill in the art.

The C₉/C₁₀ splitter may comprise a medium hydrocarbon product mixture inlet, a medium hydrocarbon stream outlet, and a heavy oil stream outlet. The C₉/C₁₀ splitter may be configured to receive the medium hydrocarbon product mixture and separate it into a medium hydrocarbon stream and a heavy oil comprising C₁₀₊ hydrocarbons.

The system may include a splitter configured to divide the light product mixture into a first recycle stream and a second stream; and the CO₂ removal unit configured to receive and remove CO₂ from the second stream. In certain embodiments, the CO₂ removal unit is coupled to the splitter and configured to remove CO₂ from the second stream. The CO₂ removal unit may include a second stream inlet and an outlet. The system may include a dryer configured to remove water from the second stream and provide a light hydrocarbon stream. The dryer may be coupled to and downstream from the CO₂ removal unit. The dryer may have a light hydrocarbon stream outlet, and an inlet coupled to the outlet on the CO₂ removal unit.

The CO₂ removal unit may comprise an amine wash and a stripper column to regenerate the amine. The amine may be regenerated in the stripper column, and the removed CO₂ may be combined with a first recycle stream and recycled by combining with the carbon source gas feed. The stripper column may have a CO₂ outlet and an amine inlet coupled to an amine outlet on the amine wash.

The system may comprise a chiller configured to cool the light hydrocarbon stream. The chiller may be coupled to the dryer and to the demethanizer column. The chiller may be configured to receive; a dried light hydrocarbon stream from the dryer and configured to supply a cooled light hydrocarbon stream to a demethanizer column. Any, chiller known for use in the art may be used herein. In certain embodiments, the chiller may reduce the temperature of light hydrocarbon stream to below about −80° C., or below about −85° C. For such an operation, an ethane chiller or ethane-propane chiller may be used.

The demethanizer column may be coupled to the chiller, or to the dryer, when present. The demethanizer column may be coupled to a deethanizer column. The pressure of the light hydrocarbon stream entering the demethanizer column may be greater than about 20 barg, or about 20 barg to about 30 barg. The pressure of the light hydrocarbon stream may be about 25 barg, or about 30 barg. The demethanizer column may operate at about 20 barg to about 30 barg, or about 22 barg to about 27 barg. The demethanizer column may operate at about 25 barg, or about 30 barg. The demethanizer column may include a cooled light hydrocarbon stream inlet, a methane outlet, and a demethanized stream outlet. The demethanized stream outlet may be coupled to a demethanized stream inlet on the deethanizer column.

The deethanizer column may be coupled to the demethanizer column and to the C₂ splitter. In certain embodiments, the deethanizer column may be configured to receive the light hydrocarbon stream from the demethanizer column. The deethanizer column may comprise a light hydrocarbon stream inlet, a deethanized stream outlet, and a C₂ hydrocarbon outlet. In other embodiments, the deethanizer column may be configured to receive the demethanized stream from the demethanizer column. The deethanizer column may comprise a demethanized stream inlet, a deethanized stream outlet, and a C₂ hydrocarbon outlet.

The C₂ splitter may have a C₂ hydrocarbon inlet, an ethylene outlet, and an ethane outlet. The C₂ splitter may be configured to separate ethylene and ethane.

The system may comprise an ethylene hydration reactor coupled to the C₂ splitter and configured to receive ethylene. The ethylene hydration reactor may comprise an ethylene hydration catalyst and may have an ethylene gas feed inlet; a water inlet; and an ethanol outlet. The ethylene hydration reactor may also comprise an unreacted ethylene outlet and a water outlet. The ethylene gas feed inlet may be coupled to an ethylene gas outlet on the C₂ splitter. The water inlet may be coupled to a water treatment unit that processes water generated from the hydrogenation reactor.

The ethylene hydration reactor may be configured such that a mixture of ethylene and steam passes through a fixed-bed reactor loaded with one or more ethylene hydration catalysts.

In further embodiments, the system comprises an ethanol purification unit coupled to the ethylene hydration reactor and configured to purify ethanol produced from ethylene hydration.

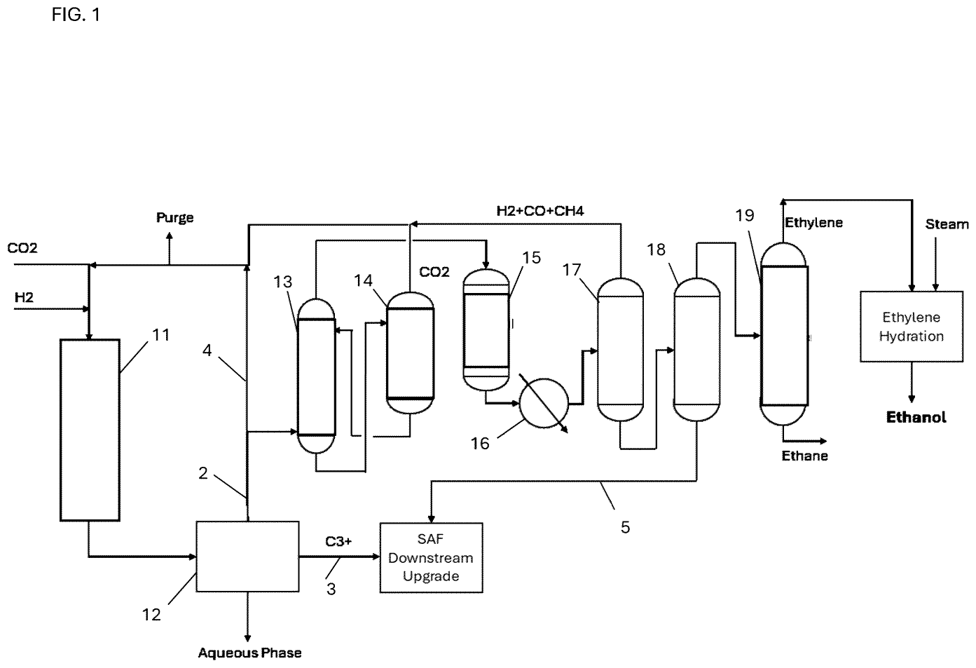

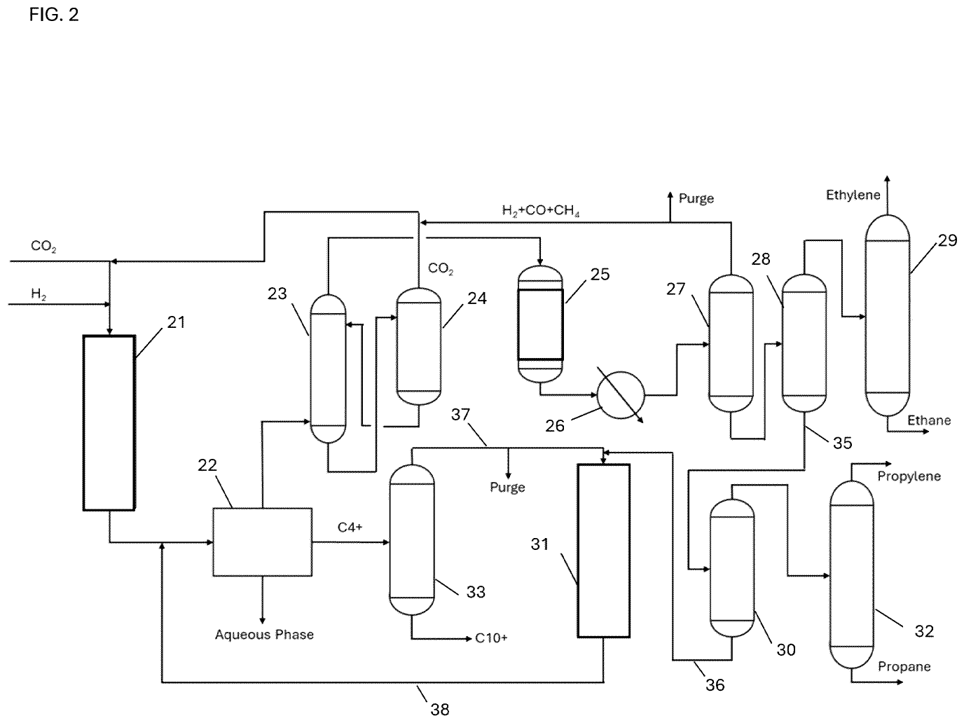

Each of the terms used in these embodiments has the same meaning as defined as above. Examples of certain systems of the invention are depicted in FIGS. 1 and 2 of the application.

FIG. 1 depicts a nonlimiting example of a system of the disclosure. As shown therein. In FIG. 1, a carbon source gas, here CO₂, and reduction gas, here H₂, are provided to a reduction reactor 11 for conversion of CO₂ to an effluent stream containing mixture of light and medium hydrocarbons, as well as other products. The effluent stream is directed to a first separator 12, where the effluent stream is divided into a light product mixture 2, a medium hydrocarbon mixture (C₃₊) 3, and an aqueous phase (e.g., water). FIG. 1 shows a first recycle stream 4 being split from the light product mixture, but alternatively, it is understood that the light product mixture need not be split before further processing and may, in other embodiments, be directed to the amine wash 13 and stripper 14 without first dividing the stream. In this figure, the split light product mixture is directed to the CO₂ removal unit, here shown as an amine wash 13 and stripper column 14 to remove CO₂ which may be recycled and combined with the carbon source gas. Once CO₂ is removed, the light hydrocarbon stream is directed to a dryer 15, shown coupled to a chiller 16, and then to a demethanizer column 17 to remove hydrogen gas, CO and methane from the stream. The demethanizer column is coupled to a deethanizer column 18 where the stream is processed for removal of C₂ hydrocarbons from the remaining components (i.e., the deethanized stream). The deethanized stream 5 may be combined with the medium hydrocarbon product stream and/or processed according to a downstream process, such as for fuel (e.g., SAF, diesel or otherwise) processing. The C₂ hydrocarbons are directed to a C₂ splitter 19 wherein ethylene is separated from ethane. Ethylene is shown as being directed to a ethylene hydration reactor for the production of ethanol. It is readily understood that in a case where ethylene is the desirable product, it may be directed to a storage unit or other end use, as desired, before conversion to ethanol.

FIG. 2 includes many of the same components as shown in FIG. 1 arranged in a similar configuration, including a reduction reactor 21, first separator 22, amine wash 23, stripper column 24, dryer 25, chiller 26, demethanizer column 27, and C₂ splitter 29. While an ethylene hydration reactor is not included in FIG. 2, one skilled in the art will readily understand that it optionally may be included in this system configuration when ethanol is a desired end product. In addition, FIG. 2 includes processing for the production of propylene and propane. In FIG. 2, the deethanizer column 28 is coupled to a depropanizer column 30, which is configured to separate C₃ hydrocarbons from the remainder of the deethanized stream 35 leaving a heavy gas stream 36, which is directed to a cracking reactor 31. The depropanizer column is coupled to a C₃ splitter 32 which separates the C₃ hydrocarbons into propylene and propane. In FIG. 2, the first separator 22 is coupled to a C₉/C₁₀ splitter 33, which is configured to remove heavy oil (C₁₀₊) from the medium hydrocarbon product mixture and to provide a medium hydrocarbon stream 37 comprising C₄-C₉ hydrocarbons to a cracking reactor 31 (with a purge). The mixed hydrocarbon product 38 from the cracking reactor is shown as recycled and combined with the effluent mixture from the reduction reactor.

### Catalysts for Conversion of Carbon Sources to Olefins and Paraffins

The systems and methods of the present disclosure may include the use of a reduction catalyst. The conversion of carbon dioxide and carbon dioxide containing mixtures can be achieved through catalytic carbon dioxide transformations, where the reduction catalyst plays the key role in the process. Reduction catalysts, as used herein may also be understood to be carbon dioxide hydrogenation catalysts, which are catalysts that enhance carbon dioxide activation and conversion, and may also control the selectivity of the hydrogenation products. The reduction catalysts are active in the conversion of a carbon source gas, such as CO₂, to hydrocarbons comprising olefins and/or paraffins.

Any known reduction catalyst may be used in accordance with this disclosure.

Transition metal catalysts, especially base metals, are particularly effective as reduction catalysts due to their high electron density, various oxidation states and rich spectrum of metal-ceramic materials, which provides enhanced carbon dioxide activations and flexible tuning of transformation pathways. In addition to the metal elements, the reduction catalyst may contain one or more additional materials, such as a binder, lubricant and/or supporting material, which can be added to optimize the forming catalyst process, metal dispersity and other chemical and physical properties.

Certain commonly known reduction catalysts contain copper, iron, cobalt, or some combination thereof. The reduction catalyst may comprise copper. Copper catalysts are known to be one of the most efficient reduction catalysts producing oxygenates as the major products. These catalysts may include copper as the core metal with various supporting elements including but not limited to zinc, zirconium, aluminum, chromium, alkali metal and alkali earth metals. The supporting element, metal alloy and metal oxide provide electronic and structure support to better tune the reactivity and selectivity of carbon dioxide hydrogenation.

The reduction catalyst may comprise iron and/or cobalt. Iron and cobalt catalysts are widely used in carbon dioxide hydrogenation, and specifically used in the Fischer-Tropsch process, for example, to form longer chain hydrocarbon and oxygenate products. Similar to the copper family, iron and cobalt catalyst may contain additional metal promoters to improve both carbon dioxide adsorption and selectivity of the hydrogenation. The metal promoter may be selected from zinc, manganese, molybdenum, copper, nickel, alkali and alkali earth metals.

Reduction catalysts of the disclosure may comprise and/or be derived from a particular metal oxide, or a combination of multiple metal oxides. One of ordinary skill in the art will appreciate that during the various catalyst preparation and activation methods known in the art, and in those exemplified herein, some or all of the oxygen atoms of the metal oxide may become bonded to other atoms in the catalyst mixture, and/or may be removed from the catalyst mixture partially or entirely during an activation step (e.g., converted to CO₂ and removed). Additionally, one of ordinary skill in the art would appreciate that for such catalysts, e.g., the reduction and/or paraffin catalysts described below, the molar ratio of oxygen relative to the total composition may vary. Further, as will be understood, when defining catalysts made from metal oxides, the molar ratios of one metal to another are defined on a metal (rather than metal oxide) basis.

The reduction catalyst may be a paraffin catalyst or an olefin catalyst. As used herein, the term “paraffin catalyst” refers to a catalyst used for the conversion of carbon sources and reduction gases to paraffins, predominantly, but which catalyst does not necessarily itself comprise paraffins. A paraffin catalyst may be selected when the desired product is paraffins. The paraffin catalyst may be used for the conversion of carbon sources and reduction gases to paraffins predominantly, as well as olefins and/or other hydrocarbons in a minority amount. As used herein, the term “olefin catalyst” refers to a catalyst used for the conversion of carbon sources and reduction gases to olefins, predominantly, but which catalyst does not necessarily itself comprise olefins. An olefin catalyst may be selected when the desired product is olefins. The olefin catalyst may be used for the conversion of carbon sources and reduction gases to olefin predominantly, as well as paraffins and/or other hydrocarbons in a minority amount.

The reduction catalyst may comprise: zinc; one or more first elements selected from iron or cobalt; and oxygen or carbon or nitrogen. The reduction catalyst may comprise: copper; zinc; one or more first elements selected from iron or cobalt; and oxygen or carbon or nitrogen. The reduction catalyst may also include aluminum. The reduction catalyst may also include one or more second elements selected from a Group V, VI, VII, VIII, IX, X, and XI metal (e.g., manganese, chromium, silver, niobium, zirconium, molybdenum, ruthenium, palladium, platinum, or nickel). The reduction catalyst may also include one or more Group IA and IIA metals.

The reduction catalyst may comprise: zinc; one or more first elements selected from iron or cobalt; oxygen or carbon or nitrogen; and aluminum. The reduction catalyst of the disclosure may comprise: zinc; one or more first elements selected from iron or cobalt; oxygen or carbon or nitrogen; aluminum; and one or more second elements selected from a Group V, VI, VII, VIII, IX, X, and XI metal (e.g., manganese, silver, niobium, zirconium, molybdenum, ruthenium, palladium, platinum, or nickel). The reduction catalyst may comprise: zinc; one or more first elements selected from iron or cobalt; oxygen or carbon or nitrogen; aluminum; and one or more Group IA and IIA metals.

The reduction catalyst may comprise: copper; zinc; one or more first elements selected from iron or cobalt; oxygen or carbon or nitrogen; and aluminum. The reduction catalyst may comprise: copper; zinc; one or more first elements selected from iron or cobalt; oxygen or carbon or nitrogen; aluminum; and one or more second elements selected from a Group V, VI, VII, VIII, IX, X, and XI metal (e.g., manganese, silver, niobium, zirconium, molybdenum, ruthenium, palladium, platinum, or nickel). The reduction catalyst may comprise: copper; zinc; one or more first elements selected from iron or cobalt; oxygen or carbon or nitrogen; aluminum; and one or more Group IA and IIA metals.

The one or more first elements may be present in an amount of about 0.5 to about 40 wt. %, about 1 to about 40 wt. %, about 0.5 to about 20 wt. %, about 5 to about 30 wt. %, about 1 to about 10 wt. %, about 10 to about 20 wt. %, about 20 to about 30 wt. %, about 25 to about 40 wt. %, about 25 to about 30 wt. %, about 22 to about 24 wt. %, about 30 to about 40 wt. %, or about 35 to about 40 wt. %, of the total amount of the copper, zinc, cobalt, iron, the optional second element, and the optional Group IA and IIA metal.

The reduction catalyst may comprise a cobalt-embedded interconnected matrix of reduced copper metal nanoparticles and alumina-modified zinc oxide. In some embodiments, the cobalt is present as cobalt oxide. In some embodiments, the copper is present as copper oxide. In some embodiments, the molar ratio of cobalt to copper to zinc (Co:Cu:Zn) is about 0.1-100 in cobalt, 0.05-4 in copper, and 0.05-2 in zinc. In some embodiments, the Co:Cu:Zn ratio is in the range of 1-2 in cobalt, 1-3 in copper, and 0.5-1 in zinc. In some embodiments, the Co:Cu:Zn ratio is approximately 1:2.5:1. In some embodiments, the zinc is preferably 0.3-1 the molar content of the copper. In some embodiments, the cobalt is preferably 0.1-1 the molar content of the copper.

The reduction catalyst may comprise an iron-embedded interconnected matrix of reduced copper metal nanoparticles and alumina-modified zinc oxide. In some embodiments, the iron is present as iron oxide. In certain embodiments, the iron oxide is magnetite (Fe₃O₄), hematite (Fe₂O₃), or a combination thereof. In further embodiments, the iron oxide is magnetite (Fe₃O₄). In yet further embodiments, the iron oxide is a combination of magnetite (Fe₃O₄) and hematite (Fe₂O₃).

In some embodiments, the copper is present as copper oxide. In some embodiments, the molar ratio of iron to copper to zinc (Fe:Cu:Zn) is about 0.1 to about 100 in iron, about 0.05 to about 4 in copper, and about 0.05 to about 4 in zinc. In some embodiments, the Fe:Cu:Zn ratio is in the range of about 0.4 to about 2 in iron, about 1 to about 3 in copper, and about 0.5-3 in zinc. In some embodiments, the Fe:Cu:Zn ratio is approximately 1:2.3:2.3. In some embodiments, the zinc is preferably about 0.3 to about 1 the molar content of the copper. In some embodiments, the iron is about 0.5 to about 5 the molar content of the copper.

The reduction catalyst may comprise one or more elements selected from a transition, or Group VI, VII, VIII, IX, X, or XI metal. In some embodiments, the reduction catalyst comprises one or more second elements selected from a Group VI metal. In some embodiments, the reduction catalyst comprises one or more second elements selected from a Group VII metal. In some embodiments, the reduction catalyst comprises one or more second elements selected from a Group VIII metal. In some embodiments, the reduction catalyst comprises one or more second elements selected from a Group IX metal. In some embodiments, the reduction catalyst comprises one or more second elements selected from a Group X metal. In some embodiments, the reduction catalyst comprises one or more second elements selected from a Group XI metal.

The one or more second elements may comprise manganese, silver, niobium, zirconium, molybdenum, ruthenium, palladium, platinum, or nickel. The one or more second elements may comprise nickel. The one or more second elements comprise silver. The one or more second elements may comprise palladium. The one or more second elements may comprise niobium. The one or more second elements may comprise manganese. The one or more second elements may comprise zirconium. The one or more second elements may comprise molybdenum.

In some embodiments, the reduction catalyst comprises the one or more second elements at a molar ratio of about 0.05 to about 4, about 0.05 to about 3, about 0.05 to about 1, about 0.05 to about 0.75, about 0.05 to about 0.5, or about 0.05 to about 0.25 relative to the one or more first elements.

In some embodiments, the reduction catalyst comprises copper at a molar ratio of about 0.5 to about 10, about 1 to about 10, about 0.5 to about 5, about 0.5 to about 2, about 1 to about 5, about 2 to about 9, about 2 to about 6, about 2 to about 4, or about 2.3 to about 8.4 relative to the one or more first elements.

In some embodiments, the reduction catalyst comprises zinc at a molar ratio of about 0.3 to about 3, about 1 to about 2.5, or about 0.4 to about 1, relative to copper.

The reduction catalyst may comprise the one or more Group IA or IIA metals. In some embodiments, the one or more Group IA or IIA metals comprise magnesium, calcium, potassium, sodium, or cesium. In some embodiments, the one or more Group IA or IIA metals consist of magnesium, calcium, potassium, sodium, or cesium. In certain embodiments, the one or more Group IA or IIA metals comprise or consist of sodium and/or cesium. In some embodiments, the reduction catalyst comprises one or more Group IA metals. The one or more Group IA or IIA metals may comprise potassium, sodium or cesium. In some embodiments, the one or more Group IA or IIA metals consist of potassium, sodium or cesium. In some embodiments, the one or more Group IA or IIA metals comprise potassium. In some embodiments, the one or more Group IA or IIA metals comprise sodium. In some embodiments, the one or more Group IA or IIA metals comprise cesium.

In some embodiments, the reduction catalyst comprises potassium at a molar ratio of about 0.05 to about 0.5, about 0.05 to about 0.1, about 0.09 to about 0.4, about 0.1 to about 0.3, or about 0.08 to about 1.0 relative to copper.

In some embodiments, the reduction catalyst comprises aluminum at a molar ratio of about 0.1 to about 10, about 0.1 to about 1, about 0.1 to about 0.2, about 0.5 to about 1 relative to copper.

The reduction catalyst may comprise one or more metal oxides selected from the group consisting of: zinc oxide, copper oxide, cobalt oxide, iron oxide, nickel oxide, and any combination thereof. The reduction catalyst may comprise alumina.

In some embodiments, the reduction catalyst comprises aluminum oxide (Al₂O₃) wherein the aluminum is present in a molar ratio of about 0.01 to about 100, about 0.1 to about 0.8, about 10 to about 50, about 30 to about 50, about 30 to about 80, about 10 to about 80, or about 5 to about 20 relative to copper. In some embodiments, the alumina can be added as a support to increase the surface area of the copper and zinc, or produced in-situ as a component of the reduction catalyst, e.g. from aluminum nitrate co-precipitation with first element, copper, and zinc precursors.

In some embodiments, the reduction catalyst comprises copper, zinc oxide, cobalt, and alumina. In some embodiments, the reduction catalyst comprises copper, zinc oxide, nickel, and alumina. In some embodiments, the reduction catalyst comprises copper, zinc oxide, iron, and alumina. In some embodiments, the reduction catalyst comprises copper, zinc oxide, cobalt, alumina, and a Group IA metal. In some embodiments, the reduction catalyst comprises copper, zinc oxide, nickel, alumina, and a Group IA metal. In some embodiments, the reduction catalyst comprises copper, zinc oxide, iron, alumina, and a Group IA metal. The molar ratios of the foregoing components may be as described above.

The reduction catalyst may comprise Cu, Zn, Al, and O. The reduction catalyst may comprise Cu, Zn, Al, O, and an alkali metal, and optionally also comprise Ni, Fe, Co, Nb, Mo, In, Se, or any combination thereof.

The elemental composition of the reduction catalyst material may be Cu(ZnO)CoA/Al₂O₃, Cu(ZnO)CoFeA/Al₂O₃, Cu(ZnO)CoNbA/Al₂O₃, Cu(ZnO)CoNiA/Al₂O₃, Cu(ZnO)CoMoA/Al₂O₃ wherein A is an alkali metal and further wherein the relative amounts of the elemental components are as described above. The elemental composition of the reduction catalyst material may be Cu(ZnO)Co/Al₂O₃, Cu(ZnO)CoFe/Al₂O₃, Cu(ZnO)CoNb/Al₂O₃, Cu(ZnO)CoNi/Al₂O₃, Cu(ZnO)CoMo/Al₂O₃, wherein the relative amounts of the elemental components are as described above. The elemental composition of the reduction catalyst material may be CuO(ZnO), Cu(ZnO)Co, Cu(ZnO)CoK, Cu(ZnO)CoFe, Cu(ZnO)CoFeK, Cu(ZnO)CoNi, Cu(ZnO)CoNiK, Cu(ZnO)CoNb, Cu(ZnO)CoNbK, Cu(ZnO)CoMo, Cu(ZnO)CoMoK on Al₂O₃, wherein the relative amounts of the elemental components are as described above.

In further aspects, provided herein are reduction catalysts comprising:
one or more metals;
optionally one or more second elements selected from copper and zinc;
optionally one or more Group VI, VII, VIII, IX, X, or XI metal additives;
optionally a Group IA or IIA metal, which acts as a promoter.

The one or more metals may be selected from cobalt, iron, nickel, indium, yttrium, a lanthanide, and combinations thereof. In certain embodiments, the one or more metals is cobalt. In other embodiments, the one or more metals is iron. In still further embodiments, the one or more metals is a combination of iron and cobalt.

The one or more metals may be present in the form of an oxide, nitride, or carbide. In certain embodiments, the one or more metals is present in the form of an iron oxide.

In further embodiments, the one or more second elements is copper. In yet further embodiments, the one or more second elements is zinc. In still further embodiments, the one or more second elements are copper and zinc. In certain embodiments, the one or more second elements is present in the form of an oxide, nitride, or carbide. In yet further embodiments, the one or more second elements is zinc oxide.

In certain embodiments, the one or more Group VI, VII, VIII, IX, X, or XI metal additives, when present, is selected from manganese, silver, niobium, zirconium, molybdenum, ruthenium, palladium, platinum, or nickel. In further embodiments, the Group IA or IIA metal, when present, are Group IA elements. In yet further embodiments, the one or more Group IA or IIA metals, when present, are magnesium, calcium, lithium, sodium, potassium, or cesium. In yet further embodiments, the Group IA or IIA metal, when present, is lithium, sodium, potassium, or cesium. In still further embodiments, the one or more second elements is present in an amount of about 0.5 to about 40 wt. % of the total amount of the one or more metals, the second element, the optional one or more Group VI, VII, VIII, IX, X, or XI metal additives, and the optional Group IA or IIA metal.

In some embodiments, the reduction catalyst comprises one or more Group VI or VII metals, such as manganese (Mn), Chromium (Cr), or a combination thereof. In some embodiments, the reduction catalyst comprises the one or more Group VI or VII metals at a molar ratio from about 0.01 to about 1.0, about 0.05 to about 0.50, about 0.1 to about 0.2, about 0.20 to about 0.50, about 0.30 to about 0.50, about 0.40 to about 0.50 relative to copper or cobalt.

In certain aspects, the reduction catalyst comprises: one or more paraffin metal oxides; optionally a support, and optionally one or more metal additives.The one or more paraffin metal oxides may be selected from cobalt oxide, iron oxide, nickel oxide, indium oxide, yttrium oxide, a lanthanide oxide, and combinations thereof. The support, when present, may comprise carbon, silica, zeolite, alumina, zirconium oxide, titanium oxide, or silica carbide. The one or more metal additives, when present, may be selected from a Group IA or IIA element, palladium, platinum, ruthenium, or combinations thereof.

In certain aspects, the present disclosure provides catalytic compositions, comprising one or more of reduction catalyst and a reduction catalyst support. The reduction catalyst support may be any suitable material that can serve as a catalyst support.

The reduction catalyst support may comprise one or more materials selected from an oxide, nitride, fluoride, silicate, or carbide of an element selected from aluminum, silicon, titanium, zirconium, cerium, magnesium, yttrium, lanthanum, zinc, tungsten, and tin. In some embodiments, the reduction catalyst support comprises 7-alumina. In certain embodiments, the reduction catalyst support is selected from carbon, silica, zeolite, alumina, zirconium oxide, titanium oxide, and silica carbide. In some embodiments, the reduction catalyst support is selected from alumina (e.g., γ-alumina), boehmite, crystalline boehmite, pseudoboehmites, gibbsites, and thermally shocked gibbsites and silicates. In some embodiments, the reduction catalyst support is an aluminum oxide that is formed in-situ as part of the reduction catalyst. In some embodiments, the reduction catalyst support is selected from, but not limited to, MgO, Al₂O₃, ZrO₂, SnO₂, SiO₂, ZnO, WO₃, and TiO₂. In some embodiments, the reduction catalyst support is selected from MgO, Al₂O₃, ZrO₂, SnO₂, SiO₂, ZnO, WO₃, silica carbide, and TiO₂.

In some embodiments, the reduction catalyst support comprises one or more carbon-based materials. In some embodiments, the carbon-based material is selected from activated carbon, carbon nanotubes, graphene, and graphene oxide.

In some embodiments, the reduction catalyst support is selected from SiAlOₓ, SO₄—ZrO₂, zirconium tungstate, tungstated-titania, and anatases (SiO₂—Al₂O₃, SiO₂—TiO₂). In further embodiments, the reduction catalyst support is an aluminum-based material such as alumina (e.g., γ-alumina), boehmite, crystalline boehmite, pseudoboehmites, gibbsites, and thermally shocked gibbsites.

In some embodiments, the reduction catalyst support is a zeolite such as Y-type zeolites, beta-zeolites, ZSM-type zeolites (e.g., ZSM-5, HZSM-5, ZSM-12, ZSM-22, ZSM-57), SAPO type zeolites (e.g., SAPO11, SAPO31, SAPO41), L zeolite (LTL), mordenite zeolites, MCM-49, MCM-22, DA-114, microcrystalline USY zeolite, microcrystalline USY zeolite, and combinations thereof. In certain embodiments, the reduction catalyst support is MCM-49. In further embodiments, the zeolites comprise additional metals such as Zn, Ga, Fe, or other transition metals. In yet further embodiments, the additional metals are present as zeolite supported metals or as isomorphous substitution in the zeolite framework.

In some embodiments, the reduction catalyst support is modified with molybdenum, chlorine, and/or sulfur.

In some embodiments, the support is a high surface area scaffold. In some embodiments, the support comprises carbon allotropes. In some embodiments, the support comprises mesoporous material, such as mesoporous silica. In such embodiments, as will be appreciated by one of ordinary skill in the art, the physical characteristics of the mesoporous material, e.g., mesopore volume and surface area may be measured using standard gas absorption measurement techniques known in the art including, for example, the Barrett-Joyner-Halenda (BJH) method for determining pore size distributions and pore volumes, and the Brunauer, Emmett and Teller (BET) method for obtaining the specific surface area (hereinafter “surface area”).

In some embodiments, the reduction catalyst support has a mesopore volume from about 0.01 to about 3.0 cc/g.

In some embodiments, the reduction catalyst support has surface area from about 1 m²/g to about 1000 m²/g. In some embodiments, the catalytic composition comprising the reduction catalyst support and a catalyst disclosed herein has a surface area from about 10 m²/g to about 1000 m²/g.

The catalytic composition may be in a form of particles having an average size from about 10 nm to about 5 μm, an average size from about 20 nm to about 5 μm, an average size from about 50 nm to about 1 μm, an average size from about 100 nm to about 500 nm, or an average size from about 50 nm to about 300 nm.

The catalytic composition may comprise about 5 wt. % to about 80 wt. %, about 5 wt. % to about 70 wt. %, about 20 wt. % to about 70 wt. %, or about 30 wt. % to about 70 wt. % of the reduction catalyst.

In some embodiments, the reduction catalyst is a nanoparticle catalyst. The particle sizes of the reduction catalyst on the surface of the scaffold may be about 1 nm to about 5 nm, about 5 nm to about 100 nm, or about 100 to about 500 nm. In some embodiments, the particles not subjected to agglomeration are about 100 nm to about 500 nm in particle size.

The reduction catalyst may comprise: iron; optionally alumina; optionally a first element selected from copper, zinc, cobalt, manganese, chromium, or combinations thereof; and optionally one or more second elements selected from Group IA and IIA metals.

In certain embodiments, the reduction catalyst further comprises an additive mixture comprising potassium, manganese, ruthenium, and MgO. In further embodiments, the reduction catalyst comprises from about 1% to about 10% by weight of the additive mixture.

The reduction catalyst may comprise a first element selected from copper, zinc, cobalt, or combinations thereof. The first element may be copper. The first element may be zinc. The first element may be cobalt. The first element may be a combination of copper, zinc, and/or cobalt.

The reduction catalyst may comprise one or more Group IA or IIA metals. The one or more Group IA or IIA metals may comprise magnesium, calcium, potassium, sodium, or cesium. The one or more Group IA or IIA metals may consist of magnesium, calcium, potassium, sodium or cesium. The one or more Group IA or IIA metals may comprise magnesium. The one or more Group IA or IIA metals may comprise calcium. The one or more Group IA or IIA metals may comprise potassium. The one or more Group IA or IIA metals may comprise sodium. The one or more Group IA or IIA metals may comprise cesium. The one or more Group IA or IIA metals may consist of magnesium. The one or more Group IA or IIA metals may consist of calcium. The one or more Group IA or IIA metals may consist of potassium. The one or more Group IA or IIA metals may consist of sodium. The one or more Group IA or IIA metals may consist of cesium.

The reduction catalyst may comprise: iron; a first element selected from K, Li, Zr, Cs, Mg, Rh, Ca, or a combination thereof; one or more second elements selected from Au, Cu, Na, Cr, Al, Ga, Mn Co, Ru, Ni, or a combination thereof, and optionally alumina.

The reduction catalyst may comprise: iron; K, Li, Zr, Cs, Mg, Rh, Ca, or a combination thereof, at a molar ratio of from 0 to about 0.20 relative to iron; Au, Cu, Na, Cr, Al, Ga, Mn, or a combination thereof, at a molar ratio from 0 to about 0.60 relative to iron; and Zn at a molar ratio from 0 to about 0.50 relative to iron.

In certain embodiments, the catalyst comprises K at a molar ratio of from 0 to about 0.20 relative to iron, and/or Na at a molar ratio from 0 to about 0.60 relative to iron.

In certain embodiments, the reduction catalyst comprises: iron; K, Cs, Mg, Rh, Ca, or a combination thereof, at a molar ratio of from 0 to about 0.20 relative to iron; Na, Cu, Cr, Mn, or a combination thereof, at a molar ratio of from 0 to about 0.60 relative to iron; Co, Ru, Ni, or a combination thereof, at a molar ratio of from 0 to about 0.50 relative to iron.

In some embodiments, the reduction catalyst comprises Co at a molar ratio of from 0 to about 0.50, or about 0.1 to about 0.2 relative to iron. In certain embodiments, the reduction catalyst comprises Co at a molar ratio of about 0.14 relative to iron, and K at a molar ratio of about 0.01 relative to iron.

The iron may be in metal form, in the form of an iron oxide, or a combination thereof. In certain embodiments, the iron is in the iron oxide form. The iron oxide may be FeO, magnetite (Fe₃O₄), hematite (Fe₂O₃), or a combination thereof. In some embodiments, the iron oxide is magnetite (Fe₃O₄). In other embodiments, the iron oxide is a combination of magnetite (Fe₃O₄) and hematite (Fe₂O₃). In other embodiments, the iron oxide is a combination of FeO, magnetite (Fe₃O₄) and hematite (Fe₂O₃).

The reduction catalyst may comprise: iron; a first element selected from copper, zinc, cobalt, or combinations thereof; and optionally one or more second elements selected from Group IA and IIA metals.

The reduction catalyst may also include one or more third elements selected from a Group V, VI, VII, VIII, IX, X, and XI metal (e.g., manganese, chromium, silver, niobium, zirconium, molybdenum, ruthenium, palladium, platinum, or nickel).

The reduction catalyst may include: iron; and the first element being zinc. One or both of the iron and zinc may be present in oxide or carbide forms. The iron oxide may be in the form of FeO, Fe₂O₃ (hematite), Fe₃O₄ (magnetite) or a combination thereof. The iron oxide may be substantially (e.g., over about 80%, or over about 90%) in the form of Fe₂O₃. The iron oxide may be substantially (e.g., over about 80%, or over about 90%) in the form of Fe₃O₄.

The reduction catalyst may comprise zinc at a molar ratio of about 0.2 to about 3 relative to iron, or about 0.3 to about 3 relative to iron. In some embodiments, the reduction catalyst comprises zinc at a molar ratio of about 0.2 to about 1 relative to iron, or about 0.4 to about 1 relative to iron. In some embodiments, the reduction catalyst comprises zinc at a molar ratio of about 1.5 relative to iron. In other embodiments, the reduction catalyst comprises zinc at a molar ratio of about 1.0 relative to iron. In certain embodiments, the reduction catalyst comprises zinc at a molar ratio of about 0.75 relative to iron, about 0.6 relative to iron, about 0.5 relative to iron, about 0.4 relative to iron, about 0.3 relative to iron, or about 0.25 relative to iron. In some embodiments, the reduction catalyst comprises zinc at a molar ratio of about 0.5 relative to iron.

The reduction catalyst may comprise a molar ratio of iron to zinc of about 1:1 to about 7:1, about 1:1 to about 6:1; about 2:2 to about 6:1, about 1:1 to about 4:1, about 1:1 to about 3:1, or about 2:1 to about 3:1. The reduction catalyst may comprise a molar ratio of iron to zinc of about 1:1 to about 4.5:1, about 1.5:1 to about 3.5:1, about 1.5:1 to about 3:1, or about 1.5:1 to about 2.5:1. The reduction catalyst may comprise a molar ratio of iron to zinc of about 2:1.

In some embodiments, the reduction catalyst comprises: iron; zinc at a molar ratio of about 0.2 to about 3 relative to iron; and one or more Group IA or IIA metals.

The one or more Group IA or IIA metals may be present at a molar ratio from 0 to about 0.60 relative to iron; and Zn at a molar ratio from 0 to about 0.50 relative to iron.

The reduction catalyst may comprise K, Na, Cs, Rh, or a combination thereof at a molar ratio of about 0.01 to about 0.20, about 0.01 to about 0.10, about 0.01 to about 0.08, about 0.01 to about 0.05, or about 0.02 to about 0.4 relative to iron. In other embodiments, the reduction catalyst comprises Na at a molar ratio of about 0.01 to about 0.20, about 0.01 to about 0.10, about 0.01 to about 0.08, about 0.01 to about 0.05, or about 0.02 to about 0.4 relative to iron.

The reduction catalyst may comprise K, Na, Cs, Rh, or a combination thereof in an amount of about 0.2% to about 1.5%, or about 0.5% to about 1.0% of the total weight of iron plus the first element. In certain embodiments, the reduction catalyst comprises Na in an amount of about 0.2% to about 1.5%, or about 0.5% to about 1.0% of the total weight of iron plus the first element. In other embodiments when the first element is zinc, the reduction catalyst may comprise Na in an amount of about 0.2% to about 1.5%, or about 0.5% to about 1.0% of the total weight of iron plus zinc.

The reduction catalyst may afford a product stream having a methane selectivity of less than about 11 carbon mole %, or less than about 10 carbon mole %. The olefin catalyst may afford a product stream having a methane selectivity of about 4 carbon mole % to about 11 carbon mole %, or about 5 carbon mole % to about 10 carbon mole %. Unless specifically identified otherwise, selectivity values disclosed herein are in carbon mole %.

The reduction catalyst may afford a product stream having an olefin to paraffin ratio (O/P) of greater than about 7. The reduction catalyst may afford a product stream having an olefin to paraffin ratio (O/P) of about 7 to about 9, about 8 to about 9, or about 8.

In certain aspects, the reduction catalyst further comprises a reduction catalyst support. The reduction catalyst support may be any suitable material that can serve as a catalyst support, or any reduction catalyst support disclosed above.

In certain embodiments, the reduction catalyst comprising the reduction catalyst support is in a form of particles having an average size from about 10 nm to about 5 μm, about 20 nm to about 5 μm, about 50 nm to about 1 μm, about 100 nm to about 500 nm, or about 50 nm to about 300 nm.

In certain embodiments, the reduction catalyst comprising the reduction catalyst support comprises from about 5 wt. % to about 80 wt. %, about 5 wt. % to about 70 wt. %, about 20 wt. % to about 70 wt. %, or about 30 wt. % to about 70 wt. % of the reduction catalyst.

In certain embodiments, the reduction catalyst support is a high surface area scaffold. In further embodiments, the reduction catalyst support comprises mesoporous silica. In yet further embodiments, the reduction catalyst support comprises carbon allotropes.

In certain embodiments, the reduction catalyst is pretreated with syngas. In yet further embodiments, the reduction catalyst is pretreated with hydrogen. In still further embodiments, the reduction catalyst is heated with inert gas (including but not limited to nitrogen gas, argon) before the production of ethanol and/or light olefins.

Reduction catalysts disclosed herein have improved selectivity for olefins and/or paraffins over methane and improved means for adjusting the olefin to paraffin ratio.

By using a reduction catalyst disclosed herein with carbon conversion, a carbon source gas may be converted into a hydrocarbon mixture comprising olefins and paraffins. The hydrocarbon mixture may have an olefin to paraffin ratio (O/P) of greater than about 7. The reduction catalyst may afford a product stream having an olefin to paraffin ratio (O/P) of about 7 to about 9, about 8 to about 9, or about 8.

The reduction catalyst may comprise iron and zinc, one or more second elements selected from Group IA, IIA, and X metals, and a binder. When the reduction catalyst includes a binder, it may also be referred to as a formed reduction catalyst. The reduction catalyst may comprise iron and zinc; optionally alumina; optionally a first element selected from copper, cobalt, manganese, chromium, or combinations thereof, optionally one or more second elements selected from Group IA, IIA, and X metals; and a binder.

The reduction catalyst may comprise a first element selected from copper, cobalt, or combinations thereof. The first element may be copper. The first element may be cobalt. The first element may be a combination of copper, and/or cobalt. The reduction catalyst may be free of a first element selected from copper, cobalt, or combinations thereof.

The reduction catalyst may comprise the second element selected from one or more Group IA or IIA metals. The one or more Group IA or IIA metals may comprise magnesium, calcium, potassium, sodium, cesium, rubidium, or any combination thereof. The one or more Group IA or IIA metals may consist of magnesium, calcium, potassium, sodium, cesium, or rubidium. The one or more Group IA or IIA metals may comprise magnesium. The one or more Group IA or IIA metals may comprise calcium. The one or more Group IA or IIA metals may comprise potassium. The one or more Group IA or IIA metals may comprise sodium. The one or more Group IA or IIA metals may comprise cesium. The one or more Group IA or IIA metals may comprise rubidium. The one or more Group IA or IIA metals may consist of magnesium. The one or more Group IA or IIA metals may consist of calcium. The one or more Group IA or IIA metals may consist of potassium. The one or more Group IA or IIA metals may consist of sodium. The one or more Group IA or IIA metals may consist of cesium. The one or more Group IA or IIA metals may consist of rubidium.

The reduction catalyst may comprise the second element being a Group X metal. The Group X metal may be selected from palladium, platinum, iridium, nickel, and rhodium. The Group X metal may be platinum. The Group X metal may be palladium. The Group X metal may be nickel.

The reduction catalyst may also include one or more third elements selected from a Group V, VI, VII, VIII, IX, and XI metal (e.g., manganese, chromium, silver, niobium, zirconium, molybdenum, ruthenium). The reduction catalyst may include manganese. The reduction catalyst may include silver. The reduction catalyst may be free of a third element selected from a Group V, VI, VII, VIII, IX, and XI metal.

The reduction catalyst may comprise the Group IA, IIA, or X metal at about 0.1 wt % to about 60 wt % of the total weight of iron, zinc, and Group IA, IIA, or X metal. The reduction catalyst may comprise the Group IA, IIA, or X metal at about 0.1 wt % to about 20 wt %, about 0.1 wt % to about 10 wt %, about 0.1 wt % to about 5 wt %, about 0.1 wt % to about 2 wt %, about 0.4 wt % to about 1.5 wt %, or about 0.5 wt % to about 1.5 wt % of the total weight of iron, zinc, and Group IA, IIA, or X metal. The reduction catalyst may comprise a Group IA metal at about 0.1 wt % to about 60 wt % of the total weight of iron, zinc, and Group IA metal. The reduction catalyst may comprise the Group IA metal at about 0.1 wt % to about 20 wt %, about 0.1 wt % to about 10 wt %, about 0.1 wt % to about 5 wt %, about 0.1 wt % to about 2 wt %, about 0.4 wt % to about 1.5 wt %, or about 0.5 wt % to about 1.5 wt % of the total weight of iron, zinc, and Group IA metal.

The reduction catalyst may comprise Na, Mn, K, Cs, Li, Rb at a molar ratio from 0 to about 0.60 relative to iron. In certain embodiments, the reduction catalyst comprises: iron; K, Cs, Mg, Rh, Ca, or a combination thereof, at a molar ratio of from 0 to about 0.20 relative to iron; Na, Cu, Cr, Mn, or a combination thereof, at a molar ratio of from 0 to about 0.60 relative to iron; and/or Co, Ru, Ni, or a combination thereof, at a molar ratio of from 0 to about 0.50 relative to iron.

The iron may be in metal form, in the form of an iron oxide, or a combination thereof. In certain embodiments, the iron is in the iron oxide form. The iron oxide may be FeO, magnetite (Fe₃O₄), hematite (Fe₂O₃), or a combination thereof. In some embodiments, the iron oxide is magnetite (Fe₃O₄). In other embodiments, the iron oxide is a combination of magnetite (Fe₃O₄) and hematite (Fe₂O₃). In other embodiments, the iron oxide is a combination of FeO, magnetite (Fe₃O₄) and hematite (Fe₂O₃).

The reduction catalyst may include: iron and zinc, with one or both of the iron and zinc being present in oxide or carbide forms. The iron oxide may be in the form of FeO, Fe₂O₃ (hematite), Fe₃O₄ (magnetite) or a combination thereof. The iron oxide may be substantially (e.g., over about 80%, or over about 90%) in the form of Fe₂O₃. The iron oxide may be substantially (e.g., over about 80%, or over about 90%) in the form of Fe₃O₄.

The reduction catalyst may comprise zinc at a molar ratio of about 0.2 to about 3 relative to iron, or about 0.3 to about 3 relative to iron. In some embodiments, the reduction catalyst comprises zinc at a molar ratio of about 0.2 to about 1 relative to iron, or about 0.4 to about 1 relative to iron. In some embodiments, the reduction catalyst comprises zinc at a molar ratio of about 1.5 relative to iron. In other embodiments, the reduction catalyst comprises zinc at a molar ratio of about 1.0 relative to iron. In certain embodiments, the reduction catalyst comprises zinc at a molar ratio of about 0.75 relative to iron, about 0.6 relative to iron, about 0.5 relative to iron, about 0.4 relative to iron, about 0.3 relative to iron, or about 0.25 relative to iron. In some embodiments, the reduction catalyst comprises zinc at a molar ratio of about 0.5 relative to iron.

The reduction catalyst may comprise a molar ratio of iron to zinc of about 1:1 to about 7:1, about 1:1 to about 6:1; about 2:2 to about 6:1, about 1:1 to about 4:1, about 1:1 to about 3:1, or about 2:1 to about 3:1. The reduction catalyst may comprise a molar ratio of iron to zinc of about 1:1 to about 4.5:1, about 1.5:1 to about 3.5:1, about 1.5:1 to about 3:1, or about 1.5:1 to about 2.5:1. The reduction catalyst may comprise a molar ratio of iron to zinc of about 2:1.

In some embodiments, the reduction catalyst comprises: iron; zinc at a molar ratio of about 0.2 to about 6 relative to iron; and one or more Group IA and IIA metals. The one or more Group IA and IIA metals may be present at a molar ratio from 0 to about 0.60 relative to iron; and Zn at a molar ratio from 0 to about 0.50 relative to iron. In some embodiments, the reduction catalyst comprises: iron; zinc at a molar ratio of about 0.2 to about 6 relative to iron; and one or more Group IA, IIA, and X metals. The one or more Group IA, IIA, and X metals may be present at a molar ratio from 0 to about 0.60 relative to iron; and Zn at a molar ratio from 0.2 to about 3 relative to iron.

The reduction catalyst may comprise K, Na, Cs, Rh, Rb, Mn, Li, Pt, Pd, Ru, Cu, Mo, Ce, or a combination thereof at a molar ratio of about 0.01 to about 0.20, about 0.01 to about 0.10, about 0.01 to about 0.08, about 0.01 to about 0.05, or about 0.02 to about 0.4 relative to iron. In other embodiments, the reduction catalyst comprises Na or K at a molar ratio of about 0.01 to about 0.20, about 0.01 to about 0.10, about 0.01 to about 0.08, about 0.01 to about 0.05, or about 0.02 to about 0.4 relative to iron.

The reduction catalyst may comprise K, Na, Cs, Rh, Rb, Mn, Li, Pt, Pd, Ru, Cu, Mo, Ce, or a combination thereof in an amount of about 0.1 wt % to about 10 wt %, about 0.2% to about 10%, about 0.1% to about 2%, about 0.5% to about 5%, about 0.2% to about 1.5%, or about 0.5% to about 1.0% of the total weight of iron plus zinc. In certain embodiments, the reduction catalyst comprises Na or K in an amount of about 0.2% to about 10%, about 0.5% to about 5%, about 0.5% to about 3%, about 0.5% to about 1%, or about 1% to about 5% of the total weight of iron plus zinc. In other embodiments the reduction catalyst may comprise Na in an amount of about 0.2% to about 10%, about 0.5% to about 5%, about 0.5% to about 3%, about 0.5% to about 1%, or about 1% to about 5% of the total weight of iron plus zinc.

The reduction catalyst may comprise iron, zinc and one or more Group IA or IIA metals, having a molar ratio of iron to zinc of about 1:1 to about 4.5:1, about 1.5:1 to about 3.5:1, about 1.5:1 to about 3:1, or about 1.5:1 to about 2.5:1; and the one or more Group IA or IIA metals present at about 0.5% to about 1.0% of the total weight of iron plus zinc.

The reduction catalyst may comprise iron, zinc, and the one or more Group IA, IIA or X metals being sodium, lithium, platinum, cesium, rubidium, manganese, or potassium, having a molar ratio of iron to zinc of about 1.5:1 to about 2.5:1; and the Na, Li, Rb, Mn, Cs, Pt, or K present at about 0.5% to about 1.0% of the total weight of iron plus zinc. The reduction catalyst may comprise iron, zinc, and the one or more Group IA or IIA metals being sodium or potassium, having a molar ratio of iron to zinc of about 1.5:1 to about 2.5:1; and the Na or K present at about 0.5% to about 1.0% of the total weight of iron plus zinc.

In certain aspects, the reduction catalysts further comprise a reduction catalyst support. The reduction catalyst support may be any suitable material that can serve as a catalyst support.

In some embodiments, the catalyst support comprises one or more materials selected from an oxide, nitride, fluoride, silicate, or carbide of an element selected from aluminum, silicon, titanium, zirconium, cerium, magnesium, yttrium, lanthanum, zinc, and tin. In further embodiments, the catalyst support comprises one or more materials selected from an oxide, nitride, fluoride, silicate, or carbide of an element selected from aluminum, silicon, titanium, zirconium, cerium, magnesium, yttrium, lanthanum, zinc, iron, and tin. In some preferred embodiments, the catalyst support comprises 7-alumina. In certain embodiments, the catalyst support is selected from carbon, silica, zeolite, alumina, zirconium oxide, titanium oxide, and silica carbide. In certain embodiments, the additional support is selected from carbon, silica, zeolite, alumina, iron oxide, zirconium oxide, titanium oxide, and silica carbide. In some embodiments, the catalyst support is an aluminum oxide that is formed in-situ as part of the catalyst. In some embodiments, the catalyst support is selected from, but not limited to, Al₂O₃, ZrO₂, SnO₂, SiO₂, ZnO, and TiO₂. In some embodiments, the catalyst support is selected from Al₂O₃, ZrO₂, SnO₂, SiO₂, ZnO, and TiO₂. In some embodiments, the catalyst support is selected from Al₂O₃, ZrO₂, SnO₂, SiO₂, ZnO, Fe₂O₃, Fe₃O₄, FeO, and TiO₂.

In some embodiments, the reduction catalyst support comprises one or more carbon-based materials. In some embodiments, the carbon-based material is selected from activated carbon, carbon nanotubes, graphene, and graphene oxide.

In some embodiments, the reduction catalyst support is selected from SiAlOₓ, SO₄—ZrO₂, zirconium tungstate, tungstated-titania, and anatases (SiO₂—Al₂O₃, SiO₂—TiO₂). In further embodiments, the reduction catalyst support is an aluminum-based material such as alumina (e.g., γ-alumina), boehmite, crystalline boehmite, pseudoboehmites, gibbsites, and thermally shocked gibbsites.

In some embodiments, the reduction catalyst support is a zeolite such as Y-type zeolites, beta-zeolites, ZSM-type zeolites (e.g., ZSM-5, HZSM-5, ZSM-12, ZSM-22, ZSM-57), SAPO type zeolites (e.g., SAPO11, SAPO31, SAPO41), L zeolite (LTL), mordenite zeolites, MCM-49, MCM-22, DA-114, microcrystalline USY zeolite, microcrystalline USY zeolite, and combinations thereof. In certain embodiments, the reduction catalyst support is MCM-49. In further embodiments, the zeolites comprise a modifier such as Zn, Ga, Fe, or other transition metals. In yet further embodiments, the modifier is present as zeolite supported metals or as isomorphous substitution in the zeolite framework.

In some embodiments, the reduction catalyst support is modified with molybdenum, chlorine, and/or sulfur.

In certain embodiments, the reduction catalyst support is a mesoporous material. In such embodiments, as will be appreciated by one of ordinary skill in the art, the physical characteristics of the mesoporous material, e.g., mesopore volume and surface area may be measured using standard gas absorption measurement techniques known in the art including, for example, the Barrett-Joyner-Halenda (BJH) method for determining pore size distributions and pore volumes, and the Brunauer, Emmett and Teller (BET) method for obtaining the specific surface area (hereinafter “surface area”). In further embodiments, the reduction catalyst support has a mesopore volume from about 0.01 to about 3.0 cc/g.

In certain embodiments, the reduction catalyst support has surface area from about 1 m²/g to about 1000 m²/g. In certain embodiments, the reduction catalyst comprising the reduction catalyst support has a surface area from about 10 m²/g to about 1000 m²/g.

In certain embodiments, the reduction catalyst comprises the reduction catalyst support in a form of particles having an average size from about 10 nm to about 5 μm, about 20 nm to about 5 μm, about 50 nm to about 1 μm, about 100 nm to about 500 nm, or about 50 nm to about 300 nm.

In certain embodiments, the reduction catalyst comprises the reduction catalyst support in an amount from about 5 wt. % to about 80 wt. %, about 5 wt. % to about 70 wt. %, about 20 wt. % to about 70 wt. %, or about 30 wt. % to about 70 wt. % of the reduction catalyst.

In certain embodiments, the reduction catalyst support is a high surface area scaffold. In further embodiments, the reduction catalyst support comprises mesoporous silica. In yet further embodiments, the reduction catalyst support comprises carbon allotropes.

In certain embodiments, the reduction catalyst is a nanoparticle catalyst. In further embodiments, the particle sizes of the reduction catalyst on the surface of the scaffold are about 1 nm to about 5 nm, about 5 nm to about 100 nm, or about 100 to about 500 nm. In certain embodiments, the particles not subjected to agglomeration are 100-500 nm in particle size.

In certain embodiments, the reduction catalyst is pretreated with syngas. In yet further embodiments, the reduction catalyst is pretreated with hydrogen. In still further embodiments, the reduction catalyst is heated with inert gas (including but not limited to nitrogen gas, argon) before the production of ethanol and/or light olefins.

The reduction catalyst may include a binder. The binder may be any binder known for use in the art. The binder may be selected from the group consisting of: boehmite (e.g., PURAL® TH 100, PURAL® TH 80, PURAL® TH 200, PURAL® 200), silica-alumina hydrate (e.g., SIRAL® 1, SIRAL® 5, SIRAL® 10, SIRAL® 20, SIRAL® 40), aluminate (e.g., sodium-aluminate), silica (e.g., silicates, such as potassium-silicate and sodium-silicate, LUDOX®) pseudoboehmite alumina (e.g., VERSAL® V-250), bentonite clay, montmorillinite clay, tungsten, zirconate, or any combination thereof.

The binder may be present in an amount of about 0.1% to about 60% by weight, about 5% to about 40%, or about 10% to about 30% by weight of the total catalyst composition. In certain embodiments, the binder is present in about 0.1% to about 30%, about 0.1% to about 20%, about 1% to about 30%, about 1% to about 20%, about 5% to about 25%, about 5% to about 20%, about 10% to about 20%, about 5% to about 15%, or about 15% to about 25% by weight of the total catalyst composition.

The binder may contain a promoter element selected from Na, K, Cs, Li, Rb, or a combination thereof. It was found that a promoter in the binder improves catalyst performance, e.g., activity, selectivity and stability; by maintaining the promoter level constant on the active metal components. In particular, the benefits of doping the binder include:
improves methane selectivity;
   the acidity improves hydrocarbon yield;
creates meso-porosity to the formed catalyst which may improve product selectivity; and
   reduces metal leaching.

The binder may be heterogeneous, amorphous or micro-porous materials. In certain embodiments, the binder may be selected from the group consisting of: sodium-aluminate, potassium-silicate, sodium-silicate, and any combination thereof. The binder may be selected from Na-aluminate, K-aluminate, Na-silicate, K-silicate, Na-zirconate, K-zirconate, Na-tungsten, K-tungsten or a combination thereof.

When a promoter is added to the binder, performance (e.g., in terms of SC₁ and SC₅₊) of the catalyst improves significantly. When comparing performance of a catalyst having a binder without a promoter to the same catalyst and binder with a promoter, SC₁ may improve (that is, decrease) by about 20% to about 65%, about 30% to about 55%, about 30% to about 40%, or about 45% to about 55%. When comparing performance of a catalyst having a binder without a promoter to the same catalyst and binder with a promoter, SCs may improve (that is, increase) by about 25% to about 75%, 30% to about 50%, about 50% to about 75%, or about 55% to about 65%. For example, the foregoing comparisons may be between a non-doped silicate binder and a doped (with promoter) silicate binder, or between a non-doped alumina binder and a doped (with promoter) silicate binder.

A binder may be preferably selected that minimizes or does not form any strong metal support interactions with the active metal(s) because forming such interactions would inhibit the catalytic properties of the active metal. A preferred binder may bond the small active metal particles together and form a sizeable extrudate/pellets (1-5 mm). These extrudates/pellets are suited for application in industrial reactors. They also have better handling properties and avoid pressure drops in large scale reactors.

The binder disclosed herein reduces metal leaching which improves the catalyst life span. With a powder, the surface area is very large and so by forming an extrudate with a binder, thermal shock in large scale reactors may be reduced.

In certain embodiments, when the reduction catalyst comprises iron oxide and zinc oxide, and a Group IA or IIA metal, and when the first carbon source gas and first reduction gas are fed into the reduction reactor, the iron oxide reacts to be in an active form selected from the group consisting of: FeₓO_{y}, FeₓC_{y}, and any combination thereof, where x is 1-3 and y is 0-4. The active form acts to convert CO₂ to hydrocarbons selected from the group consisting of: olefins, paraffins, oxygenates, and any combination thereof.

The reduction catalyst used herein may be, but is not limited to, those reduction catalysts disclosed in co-owned U.S. Patent Application Publication No. 2024/0124792, published on Apr. 18, 2024, titled: SYNTHETIC FUELS, AND METHODS AND APPARATUS FOR PRODUCTION THEREOF; in co-owned International Publication No. WO 2025/096891, published on May 8, 2025, titled: SYSTEMS, METHODS, AND CATALYSTS FOR THE PRODUCTION OF SUSTAINABLE AVIATION FUEL; or in co-owned International Publication No. WO 2025/096928, published on May 8, 2025, titled: REDUCTION CATALYSTS, PROPERTIES THEREOF, AND METHODS OF MAKING AND USING THE SAME. The entire contents of each of the foregoing patent documents is incorporated by reference herein.

### Catalysts for Cracking

The systems and methods of the present disclosure can use any suitable cracking catalyst, including those known in the art useful for cracking C₄ or naphtha range hydrocarbons and selective for producing light olefins.

Any suitable cracking catalysts known in the art may be used in these processes. However, the particular embodiments set forth below are provided both to exemplify the use of such catalysts and to identify catalysts particularly well-suited for use in conjunction with the other features of the systems and methods disclosed herein.

The cracking catalyst may comprise a metal, an optionally modified zeolite, or an optionally modified metal oxide.

The zeolite may be selected from Y-type zeolites, beta-zeolites, ZSM-type zeolites (e.g., ZSM-5, HZSM-5, ZSM-11, ZSM-12, ZSM-22, ZSM-57), SAPO type zeolites (e.g., SAPO11, SAPO31, SAPO41), L zeolite (LTL), mordenite zeolites, MCM-49, MCM-22, PSH-3, DA-114, microcrystalline USY zeolite, microcrystalline USY zeolite, and any combination thereof. The zeolite may be a ZSM-type zeolite. The zeolite may be a ZSM-type zeolite selected from ZSM-5, HZSM-5, ZSM-11, ZSM-12, ZSM-22, ZSM-57, and any combination thereof. The zeolite may be modified with molybdenum, chlorine, and/or sulfur.

The metal oxide may be selected from cobalt oxide, magnesium oxide, iron oxide, nickel oxide, indium oxide, yttrium oxide, a lanthanide oxide, and combinations thereof. The metal oxide may be modified with a metal selected from zinc, iron, lithium, and any combination thereof. The modified metal oxide may be selected from metal modified MgO, such as, but not limited to, ZN/MgO, Li/MgO or Fe/MgO.

The metal may be a hydrocracking metal selected from Zn, La, Fe, Al, Pd, Pt, Ni, Co, Co—W, Ni—W, Ni—Mo, or any combination thereof. The cracking catalyst may comprise a hydrocracking metal, such as Pd, Pt, Ni, Co, Co—W, Ni—W, and Ni—Mo, and a hydrocracking support.

The cracking catalyst may comprise a hydrocracking support. The hydrocracking support may be any suitable material that can serve as a catalyst support.

The hydrocracking support may comprise one or more materials selected from an oxide, nitride, fluoride, silicate, or carbide of an element selected from aluminum, silicon, titanium, zirconium, cerium, magnesium, yttrium, lanthanum, zinc, tungsten, and tin. In some embodiments, the hydrocracking support comprises γ-alumina. In certain embodiments, the hydrocracking support is selected from carbon, silica, zeolite, alumina, zirconium oxide, titanium oxide, and silica carbide. In some embodiments, the hydrocracking support is selected from alumina (e.g., γ-alumina), boehmite, crystalline boehmite, pseudoboehmites, gibbsites, and thermally shocked gibbsites. In some embodiments, the hydrocracking support is an aluminum oxide that is formed in-situ as part of the reduction catalyst. In some embodiments, the hydrocracking support is selected from, but not limited to, MgO, Al₂O₃, ZrO₂, SnO₂, SiO₂, ZnO, WO₃, and TiO₂. In some embodiments, the hydrocracking support is selected from MgO, Al₂O₃, ZrO₂, SnO₂, SiO₂, ZnO, WO₃, silica carbide, and TiO₂.

In some embodiments, the hydrocracking support comprises one or more carbon-based materials. In some embodiments, the carbon-based material is selected from activated carbon, carbon nanotubes, graphene, and graphene oxide.

In some embodiments, the hydrocracking support is selected from SiAlOₓ, SO₄—ZrO₂, zirconium tungstate, tungstated-titania, and anatases (SiO₂—Al₂O₃, SiO₂—TiO₂). In further embodiments, the hydrocracking support is an aluminum-based material such as alumina (e.g., γ-alumina), boehmite, crystalline boehmite, pseudoboehmites, gibbsites, and thermally shocked gibbsites.

In some embodiments, the hydrocracking support is a zeolite such as Y-type zeolites, beta-zeolites, ZSM-type zeolites (e.g., ZSM-5, HZSM-5, ZSM-12, ZSM-22, ZSM-57), SAPO type zeolites (e.g., SAPO11, SAPO31, SAPO41), L zeolite (LTL), mordenite zeolites, MCM-49, MCM-22, PSH-3, DA-114, microcrystalline USY zeolite, microcrystalline USY zeolite, and combinations thereof. In further embodiments, the zeolites comprise a modifier such as Zn, Ga, Fe, or other transition metals. In yet further embodiments, the modifier is present as a zeolite supported metal or as isomorphous substitution in the zeolite framework.

### Carbon Source Gas and Reduction Gas

The systems and methods of the present disclosure can be designed to utilize any combination of suitable reduction gas and suitable carbon source gas. Said carbon source and reduction gas may, in certain embodiments, be provided into the requisite reaction vessels separately, or they may in certain embodiments be pre-mixed (e.g., the reduction gas feed and the carbon source gas feed can, in some embodiments refer to the same physical feature) to provide a single feed stream comprising both a carbon source gas and a reduction gas, which is coupled to the appropriate reactor.

Additionally, a single gas feed comprising the reduction gas feed and the carbon source gas feed may be pre-mixed to provide a single feed stream comprising both a carbon source gas and a reduction gas coupled to the reduction reactor.

In certain embodiments, the single gas feed may include CO₂, H₂, CO, C₂, C₃, CH₄, and any combination thereof. The feed stream may contain H₂/CO₂, in a range of about 10% to about 95%, and each of CO, C₂, C₃, and CH₄ in the range of about 0% to about 65%. The source of CO, C₂, C₃, and/or CH₄ may be from a recycle stream(s) or may be introduced in the fresh feed stream.

The reduction gas may be selected from H₂, a hydrocarbon, synthesis gas (CO/H₂), or from a gas that is, or is derived from, flare gas, waste gas, or natural gas. In certain embodiments, reduction gas may be selected from H₂. In further embodiments, the reduction gas is synthesis gas. In yet further embodiments, reduction gas is a hydrocarbon, such as CH₄, ethane, propane, or butane. In still further embodiments, the reduction gas is derived from, flare gas, waste gas, or natural gas. In certain embodiments, the reduction gas is CH₄.

The carbon source gas may comprise CO₂. The carbon source gas may comprise CO. The carbon source gas may be CO₂. The carbon source gas may be CO.

As will be understood by those of skill in the art, the flow rate of carbon source gas and/or reduction gas, or various product mixtures through the paraffin and/or aromatic reactors (or elsewhere in the systems and methods) can be adjusted as needed to afford the desired product output characteristics.

Additionally, as will be understood by those of skill in the art, the carbon source gases and the reduction gases may be provided in any suitable ratio that affords the desired product output characteristics. In certain embodiments, the molar ratio of the reduction gas to the carbon source gas is from about 10:1 to about 1:10, or about 5:1 to about 0.5:1.

### Examples

The invention now being generally described. It will be more readily understood by reference to the following examples which are included merely for purposes of illustration of certain aspects and embodiments of the present invention and are not intended to limit the invention.

### Example 1: Synthesis of Reduction Catalyst 1

A copper oxide, zinc oxide, iron oxide was first prepared by coprecipitation using a metal nitrate precursor solution. The mixture was then reacted with sodium carbonate at the same concentration at 338 K. The end point pH of the coprecipitation was at 9. The resulting slurry was then aged while continuously stirring at 353 K for 1 hour. The precipitate was then obtained by using vacuum filtration. Excess sodium was removed by water washing. The resulting solid was dried at 393 K for 4 hours and then ground to a fine powder. The promotors were then added via incipient wetness impregnation method. The solid was dried and calcined at 623 K for 6 hours.

### Example 2: Synthesis of Reduction Catalyst 2

A zinc oxide, iron oxide was first prepared by coprecipitation using a metal nitrate precursor solution. The was then reacted with sodium carbonate at the same concentration at 338 K. The end point pH of the coprecipitation was at 9. The resulting slurry was then aged while continuously stirring at 353 K for 1 hour. The precipitate was then obtained by using vacuum filtration. Excess sodium was removed by water washing. The resulting solid was dried at 393 K for 4 hours and then ground to a fine powder. The promotors were then added via incipient wetness impregnation method. The solid was then dried and calcined at 623 K for 6 hours.

### Example 3: General Procedure for Conversion of CO₂ to Ethanol and Hydrocarbons with Reduction Catalyst 1

CO₂ hydrogenation was carried out in a fixed bed flow reactor. The reactor was loaded with 5 g of the Reduction Catalyst 1 from Example 1. The catalyst was reduced in situ in a hydrogen atmosphere. For the reaction phase, a feed containing CO₂ and H₂ at a volume ratio of 1:2 was introduced into the reactor. The reactor was heated to 275° C. with pressure increase to 750 psig, at GHSV of 2000. As shown in Table 1, the process achieved a CO₂ conversion of 29%, with CO selectivity of 18%, ethylene selectivity of 3%, and ethanol selectivity of 13%. The liquid hydrocarbon selectivity was 25% with an acetic acid selectivity of 0.2%. The selectivity refers to molar selectivity, which is calculated as: #moles of product generated divided by #moles of converted CO₂.

Acetic acid may be understood as an intermediate to form ethanol so the lower acetic acid usually means higher ethanol selectivity. In this example, lower acetic acid yield shows better results. Table 1 presents a summary of the conversion and performance of Reduction Catalyst 1.

TABLE 1

| | Carbon selectivity in carbon mole % | | | | |
|---|---|---|---|---|---|
| Single pass CO₂ | | | | Acetic | Liquid |
| conversion (%) | CO | Ethanol | Ethylene | acid | Hydrocarbons |
| 29% | 18% | 13% | 3% | 0.2% | 25% |

### Example 4: General Procedure for Conversion of CO₂ to Ethanol and Hydrocarbons with Reduction Catalyst 2

CO₂ hydrogenation was carried out in a fixed bed flow reactor. The reactor is loaded with 5 g of Reduction Catalyst 2 from Example 2. The catalyst was reduced in situ in a hydrogen atmosphere. For the reaction phase, a feed containing CO₂ and H₂ at a volume ratio of 1:2 was introduced. The reactor was heated to 320° C. with pressure increase from 100 psig to 450 psig, at GHSV of 4000. The process gave a CO₂ conversion of 27%, with CO selectivity is 18% and ethylene selectivity of 6% and ethanol selectivity of 4%. The liquid hydrocarbon selectivity is 25% with an acetic acid selectivity at 1%. Table 2 presents a summary of the conversion and performance of Reduction Catalyst 2.

TABLE 2

| | Carbon selectivity in carbon mole % | | | | |
|---|---|---|---|---|---|
| Single pass CO₂ | | | | Acetic | Liquid |
| conversion (%) | CO | Ethanol | Ethylene | acid | Hydrocarbons |
| 27% | 18% | 6% | 6% | 1% | 25% |

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of making ethylene, ethanol or both ethylene and ethanol from a carbon source gas comprising:
contacting a reduction gas and the carbon source gas with a reduction catalyst to afford an effluent stream comprising a medium hydrocarbon product mixture and a light product mixture, wherein the light product mixture comprises one or more C₁₋₂ hydrocarbons, and CO₂;
separating the light product mixture from the medium hydrocarbon product mixture;
removing CO₂ from the light product mixture to provide a light hydrocarbon stream comprising the one or more C₁₋₂ hydrocarbons;
separating the light hydrocarbon stream into a C₂ hydrocarbon stream and a deethanized stream;
separating the C₂ hydrocarbon stream into ethylene and ethane; and
optionally contacting the ethylene with an ethylene hydration catalyst to make ethanol.

2. The method of claim 1, wherein the light product mixture further comprises CO, and H₂.

3. The method of claim 1, comprising splitting a first recycle stream from the light product mixture before the step of removing CO₂.

4. The method of claim 3, wherein the first recycle stream is about 1 wt % to about 99.9 wt % of the light product mixture.

5. The method of claim 1, wherein the light hydrocarbon stream comprises methane, CO, H₂, ethane and ethylene.

6. The method of am claim 1, wherein the ethylene hydration catalyst is selected from a solid or liquid acid catalyst, or a zeolite.

7. (canceled)

8. (canceled)

9. (canceled)

10. The method of claim 1, further comprising hydrating the ethylene with water concurrently with contacting the ethylene with the ethylene hydration catalyst to make ethanol.

11. The method of claim 1, further comprising drying the light hydrocarbon stream.

12. The method of claim 1, further comprising cooling the light hydrocarbon stream.

13. The method of claim 1, further comprising:
cooling the light hydrocarbon stream, and
separating a second recycle stream from the cooled light hydrocarbon stream,
wherein the second recycle stream comprises methane, CO and H₂.

14. The method of claim 1, further comprising:
drying the light hydrocarbon stream;
cooling the dried light hydrocarbon stream; and
separating a second recycle stream from the cooled light hydrocarbon stream,
wherein the second recycle stream comprises methane, CO and H₂.

15. The method of claim 1, further comprising separating the light hydrocarbon stream into a second recycle stream and a demethanized stream, and separating the demethanized stream into the C₂ hydrocarbon stream and the deethanized stream.

16. (canceled)

17. The method of claim 1, wherein the deethanized stream is combined with the medium hydrocarbon product mixture.

18. The method of claim 1, further comprising:
separating the deethanized stream into a C₃ stream and a heavy gas stream; and
separating the C₃ hydrocarbon stream into propylene and propane.

19. The method of claim 17, further comprising: removing heavy oil from the medium hydrocarbon product mixture to provide a medium hydrocarbon stream;
contacting the medium hydrocarbon stream and the heavy gas stream with a cracking catalyst to afford a mixed hydrocarbon stream; and
combining the mixed hydrocarbon stream with the effluent stream.

20. The method of claim 1, wherein the carbon source gas is selected from the group consisting of: CO, CO₂, a hydrocarbon, and a combination thereof.

21. The method of claim 1, wherein the carbon source gas comprises CO₂.

22. The method of claim 1, comprising contacting the ethylene with an ethylene hydration catalyst to make ethanol, and purifying the ethanol.

23. A system for the production of light olefins, ethanol or both light olefins and ethanol from a carbon source gas comprising:
(i) a reduction gas feed;
(ii) a carbon source gas feed;
(iii) a reduction reactor comprising a reduction catalyst, said reduction reactor having a reduction gas feed inlet, a carbon source feed inlet, and an effluent stream outlet; wherein the reduction gas feed inlet is coupled to the reduction gas feed, and the carbon source gas feed inlet is coupled to the carbon source gas feed;
(iv) a first separator having an effluent stream inlet coupled to the effluent stream outlet, said first separator being configured to separate a light product mixture from a medium hydrocarbon product mixture;
(v) a CO₂ removal unit coupled to the first separator, having a light hydrocarbon stream outlet;
(vi) a demethanizer column configured to receive a light hydrocarbon stream from the CO₂ removal unit;
(vii) a deethanizer column coupled to the demethanizer column, and configured to provide a C₂ hydrocarbon stream and a deethanized stream; and
(viii) a C₂ splitter configured to receive the C₂ hydrocarbon stream from the deethanizer column.

24-32. (canceled)

33. A method of making light olefins from a carbon source gas comprising:
contacting a reduction gas and the carbon source gas with a reduction catalyst to afford an effluent stream comprising a medium hydrocarbon product mixture and a light product mixture, wherein the light product mixture comprises one or more C₁₋₃ hydrocarbons, and CO₂;
separating the light product mixture from the medium hydrocarbon product mixture;
removing CO₂ from the light product mixture to provide a light hydrocarbon stream comprising the one or more C₁₋₃ hydrocarbons;
separating the light hydrocarbon stream into a C₂ hydrocarbon stream and a deethanized stream;
optionally separating the C₂ hydrocarbon stream into ethylene and ethane;
separating the deethanized stream into a C₃ hydrocarbon stream and a heavy gas stream; and
separating the C₃ hydrocarbon stream into propylene and propane.

34-45. (canceled)
